(19) United States
(12) Patent Application Publication
(10) Pub. No.: US 2010/0136110 A1
(43) Pub. Date: Jun. 3, 2010

(54) GRANULAR PHARMACEUTICAL COMPOSITION FOR ORAL ADMINISTRATION

(75) Inventors: Hiroaki Tasaki, Tokyo (JP); Atsushi Maeda, Chuo-ku (JP); Takeshi Yano, Tokyo (JP); Kazuhiro Sako, Tokyo (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Astellas Pharma Inc., Tokyo (JP)

(21) Appl. No.: 12/568,257

(22) Filed: Sep. 28, 2009

Related U.S. Application Data
(60) Provisional application No. 61/101,379, filed on Sep. 30, 2008.
(60) Provisional application No. 61/101,394, filed on Sep. 30, 2008.

(51) Int. Cl. A61K 9/20; A61K 9/16

(52) U.S. Cl. 424/464; 424/497; 424/494

(57) ABSTRACT

A granular pharmaceutical composition for oral administration, wherein a drug-containing particle is coated with a coating comprising a methyl methacrylate-butyl methacrylate-dimethylaminoethyl methacrylate copolymer and a water-soluble polymer is disclosed.

### TECHNICAL FIELD

The present invention relates to a granular pharmaceutical composition for oral administration containing a drug.

More particularly, the present invention relates to a granular pharmaceutical composition for oral administration, wherein a drug-containing particle is coated with a coating comprising a methyl methacrylate-butyl methacrylate-dimethylaminoethyl methacrylate copolymer and a water-soluble polymer, and relates to a rapidly disintegrating tablet in the buccal cavity, comprising this granular pharmaceutical composition for oral administration.

Further, the present invention relates to a use of a methyl methacrylate-butyl methacrylate-dimethylaminoethyl methacrylate copolymer and a water-soluble polymer in the manufacture of a granular pharmaceutical composition for oral administration wherein a drug-containing particle is coated therewith to decrease a change in a dissolution rate after compression-molding.

Furthermore, the present invention relates to a process of manufacturing a granular pharmaceutical composition for oral administration wherein a drug-containing particle is coated to decrease a change in a dissolution rate after compression-molding.

### BACKGROUND ART

A granular pharmaceutical composition for oral administration such as granules, fine particles, or powders has a size smaller than that of tablets or capsules, and therefore, it is a formulation which is easily administered to a patient having difficulty in swallowing tablets or capsules. However, because such a granular pharmaceutical composition for oral administration has an increased specific surface area due to its small size, the drug is rapidly released in the buccal cavity after administration, and as a result, various problems are caused. For example, when a drug has an unpleasant taste, the drug rapidly released in the buccal cavity sometime renders the patient highly unpleasant to drastically decrease the drug dosing compliance of the patient.

To avoid such problems in a granular pharmaceutical composition for oral administration, it is necessary to inhibit or decrease the drug release in the buccal cavity in a certain period of time. For example, when a drug has an unpleasant taste, a method of decreasing the drug release for a certain period of time while the granular pharmaceutical composition exists in the buccal cavity may be applied.

As a formulation in which convenience in administration of a medicament is increased, an attention has been paid to a rapidly disintegrating tablet in the buccal cavity, which can be administered without water and is relatively easily administered to a patient having difficulty in swallowing.

As prior art with respect to such a rapidly disintegrating tablet in the buccal cavity, for example, a rapidly disintegrating tablet in the buccal cavity characterized by being prepared by coating and/or granulating a saccharide having a low moldability with a saccharide having a high moldability as a binder and, if a higher tablet strength is needed, further subjecting the resulting product to a humidity treatment and a drying treatment (patent literature 1); a rapidly disintegrating tablet in the buccal cavity comprising a drug, a diluent, and a saccharide having a melting point relatively lower than those of the drug and the diluent, wherein the saccharide having a low melting point is uniformly mixed in the tablet, and the drug and/or diluent particles are cross-linked with a melted and solidified product of the saccharide having a low melting point (patent literature 2); a rapidly disintegrating tablet in the buccal cavity comprising a drug, a treated starch having a degree of gelatinization of 30% to 60%, and a saccharide (patent literature 3); and the like, are known.

When, for example, a drug having a bitter taste is applied to these rapidly disintegrating tablets in the buccal cavity, for example, a method in which a drug-containing particles are prepared by spraying a core consisting of crystalline cellulose with a drug solution, and then, the prepared particles are film-coated with an appropriate polymer may be used. However, it is technically very difficult to reduce the bitter taste of the drug in the buccal cavity by this method, because the film is sometimes broken when some types of coating polymers are used or when tabletting is carried out under a high pressure, and this breakage causes a leak of the drug. By contrast, when the tabletting is carried out under a low pressure to avoid the breakage of the coated film caused by the tabletting, it is anticipated that a tablet hardness suitable for handling during production or transport cannot be obtained.

A use of acrylic polymers is known as a method of reducing an unpleasant taste.

An oral pharmaceutical composition in timed-release particle form, comprising a drug-containing core, a middle layer containing two types of water-soluble components, an insolubilizer and an insolubilizing substance, and an outer layer for controlling water penetration speed, is known, and acrylic polymers are exemplified as a material used for the layer for controlling water penetration (patent literature 4). However, when some drugs or some bases are selected, there is still room for further improvements in decreasing the initial drug dissolution and achieving the subsequent rapid drug release.

To prepare a chewable taste-masked formulation, an invention relating to a taste-masked pharmaceutical composition comprising a drug, and a polymer mixture for coating a core which contains a mixture of at least 5% by weight of a high temperature film forming polymer and at least 5% by weight of a low temperature film forming polymer is known (patent literature 5).

A film coating agent which contains methylcellulose and an acrylic polymer comprising a methacrylic ester and/or an acrylic ester as a monomer unit, and which can mask an unpleasant taste such as a bitter taste of a solid formulation by film coating and can impart an excellent dissolubility (patent literature 6).

However, patent literatures 5 and 6 do not disclose a change in dissolution before and after the compression-molding of coated particles, and therefore, a change in dissolution caused by compression-molding is anticipated.

When a granular composition coated with a methyl methacrylate-butyl methacrylate-dimethylaminoethyl methacrylate copolymer is compression-molded, its dissolution-controlling function is lost. A method of regenerating the dissolution-controlling function at the inside the compression-molded product by performing a treatment with an alcohol solvent is disclosed (patent literature 7).

As a method of compression-molding with a filler capable of absorbing damage by compression-molding, a formulation prepared by physically mixing a drug-containing coated particles with a film protecting agent having an average particle diameter of 20 μm or less, and then compression-molding the resulting mixture is disclosed. To provide a compression molded formulation having reduced damage of a coating film caused by compression-molding of drug-containing coated particles, an invention relating to a compression molded formulation comprising drug-containing coated particles and a finely granular film protecting agent having an average particle diameter of approximately 50 μm or more and an initial dissolution rate ratio of 4 or more is disclosed (patent literature 8).

However, with respect to prior art disclosed in patent literature 7 or 8, when some drugs or some bases are selected, a disadvantage that the initial drug dissolution can be decreased, but the following rapid drug release cannot be achieved, or another disadvantage that it is necessary to use a special apparatus capable of carrying out an alcohol treatment, are anticipated.

As described above, a rapidly disintegrating tablet in the buccal cavity which may be prepared by a convenient formulation and process and contains a granular pharmaceutical composition coated with a methyl methacrylate-butyl methacrylate-dimethylaminoethyl methacrylate copolymer which can be easily applied to known rapidly disintegrating tablets in the buccal cavity and reduces a change in dissolution before and after compression-molding is unknown. In the medical field, it has been desired to technically develop a rapidly disintegrating tablet in the buccal cavity containing a granular pharmaceutical composition for oral administration capable of decreasing the amount of initial drug dissolution and maintaining the subsequent rapid drug release and further capable of inhibiting or decreasing a change in drug dissolution rate after compression-molding.

### Citation List

### Patent Literature

[patent literature 1] International Publication No. WO 95/20380 (corresponding to U.S. Pat. No. 5,576,014)
[patent literature 2] International Publication No. WO 02/92057 (corresponding to U.S. Pat. No. 6,872,405)
[patent literature 3] International Publication No. WO 2008/032767
[patent literature 4] International Publication No. WO 2005/105045 (corresponding to U.S. Patent Application Publication No. 2005/0287211)
[patent literature 5] Japanese Translation Publication (Kohyo) No. 1-502589
[patent literature 6] Japanese Unexamined Patent Publication (Kokai) No. 2001-192344
[patent literature 7] Japanese Unexamined Patent Publication (Kokai) No. 2006-45134
[patent literature 8] Japanese Unexamined Patent Publication (Kokai) No. 8-333242

### SUMMARY OF INVENTION

### Technical Problem

The present invention provides a granular pharmaceutical composition for oral administration capable of inhibiting or decreasing the amount of initial drug dissolution and maintaining the subsequent rapid drug release and further capable of decreasing a change in drug dissolution rate after compression-molding, and a rapidly disintegrating tablet in the buccal cavity containing the same.

Further, the present invention provides a use of a methyl methacrylate-butyl methacrylate-dimethylaminoethyl methacrylate copolymer and a water-soluble polymer in the manufacture of a granular pharmaceutical composition for oral administration wherein a drug-containing particle is coated therewith to decrease a change in a dissolution rate after compression-molding.

Furthermore, the present invention provides a process of manufacturing a granular pharmaceutical composition for oral administration coated with a coating comprising a methyl methacrylate-butyl methacrylate-dimethylaminoethyl methacrylate copolymer and a water-soluble polymer.

### Solution to Problem

Under these circumstances, the present inventors conducted intensive studies on a coating substance capable of decreasing drug release from a core containing a drug having a bitter taste in compression-molding, it was found, in prior art, (1) that the drug release from the core was promoted under a certain tabletting pressure, and (2) that a rapid release after a certain period of time could not be achieved in a certain combination of coating components. Further, the present inventors found that the drug release from the core after compression-molding could be decreased by coating the drug-containing core with a coating comprising a methyl methacrylate-butyl methacrylate-dimethylaminoethyl methacrylate copolymer and a water-soluble polymer, and that it was possible to impart a sufficient lag time and arbitrarily control the length of the lag time, without adding an insolubilizer to the drug-containing particles. Furthermore, the present inventors found that these findings could be applied to not only a drug having a bitter taste but also a drug exhibiting a pharmaceutically adverse phenomenon accompanied by the release change, and completed the present invention.

The present invention relates to:
[1] a granular pharmaceutical composition for oral administration, wherein a drug-containing particle is coated with a coating comprising a methyl methacrylate-butyl methacrylate-dimethylaminoethyl methacrylate copolymer and a water-soluble polymer;
[2] the granular pharmaceutical composition for oral administration of [1], wherein the coating further comprises a glidant;
[3] the granular pharmaceutical composition for oral administration of [1] or [2], wherein the water-soluble polymer is one compound, or two or more compounds selected from the group consisting of hydroxypropyl cellulose, hydroxypropyl methylcellulose, methylcellulose, hydroxyethyl cellulose, povidone, copolyvidone, a polyvinyl alcohol-polyethylene glycol graft copolymer, polyvinyl alcohol, macrogol, and polyethylene oxide;
[4] the granular pharmaceutical composition for oral administration of any one of [1] to [3], wherein the water-soluble polymer is one compound, or two or more compounds selected from the group consisting of hydroxypropyl cellulose, hydroxypropyl methylcellulose, methylcellulose, and hydroxyethyl cellulose;
[5] the granular pharmaceutical composition for oral administration of any one of [1] to [4], wherein an amount of the water-soluble polymer is 1% by weight to 30% by weight with respect to an amount of the methyl methacrylate-butyl methacrylate-dimethylaminoethyl methacrylate copolymer;
[6] the granular pharmaceutical composition for oral administration of any one of [1] to [5], wherein the glidant is one compound, or two or more compounds selected from the group consisting of metal silicates, silicon dioxides, higher fatty acid metal salts, metal oxides, alkaline earth metal salts, and metal hydroxides;
[7] the granular pharmaceutical composition for oral administration of any one of [1] to [6], wherein the glidant is one compound, or two or more compounds selected from the group consisting of talc, kaolin, calcium silicate, magnesium silicate, light anhydrous silicic acid, magnesium stearate, calcium stearate, iron oxide, titanium oxide, calcium carbonate, calcium phosphate, gypsum, magnesium carbonate, aluminum hydroxide, hydrated silicon dioxide, microcrystalline cellulose, synthetic aluminum silicate, heavy anhydrous silicic acid, aluminum magnesium hydroxide, stearic acid, corn starch, magnesium aluminate metasilicate, dibasic calcium phosphate fine granulated, and glyceryl monostearate;
[8] the granular pharmaceutical composition for oral administration of any one of [1] to [7], wherein the glidant is one compound, or two or more compounds selected from the group consisting of talc, kaolin, calcium silicate, magnesium silicate, light anhydrous silicic acid, magnesium stearate, and glyceryl monostearate;
[9] the granular pharmaceutical composition for oral administration of any one of [1] to [8], wherein an amount of the glidant is 1% by weight to 500% by weight with respect to an amount of the methyl methacrylate-butyl methacrylate-dimethylaminoethyl methacrylate copolymer;
[10] the granular pharmaceutical composition for oral administration of any one of [1] to [9], wherein the water-soluble polymer is hydroxypropyl methylcellulose, and an amount of hydroxypropyl methylcellulose is 1% by weight to 30% by weight with respect to an amount of the methyl methacrylate-butyl methacrylate-dimethylaminoethyl methacrylate copolymer;
[11] the granular pharmaceutical composition for oral administration of any one of [1] to [10], wherein the drug is an acidic drug or a salt thereof;
[12] the granular pharmaceutical composition for oral administration of any one of [1] to [11], wherein the drug has an unpleasant taste;
[13] the granular pharmaceutical composition for oral administration of any one of [1] to [12], wherein (1) a coating layer containing (i) a coating consisting of a methyl methacrylate-butyl methacrylate-dimethylaminoethyl methacrylate copolymer and a water-soluble polymer, or (ii) a coating consisting of a methyl methacrylate-butyl methacrylate-dimethylaminoethyl methacrylate copolymer, a water-soluble polymer, and a glidant surrounds (2) a layer comprising a water-soluble insolubilizer and a water-soluble insolubilizing substance;
[14] a rapidly disintegrating tablet in the buccal cavity, comprising the granular pharmaceutical composition for oral administration of any one of [1] to [13];
[15] a process of manufacturing a granular pharmaceutical composition for oral administration, characterized by coating a drug-containing particle with a coating comprising a methyl methacrylate-butyl methacrylate-dimethylaminoethyl methacrylate copolymer and a water-soluble polymer;
[16] the process of [15], wherein the coating further comprises a glidant;
[17] the process of [15] or [16], wherein the water-soluble polymer is one compound, or two or more compounds selected from the group consisting of hydroxypropyl cellulose, hydroxypropyl methylcellulose, methylcellulose, hydroxyethyl cellulose, povidone, copolyvidone, a polyvinyl alcohol-polyethylene glycol graft copolymer, polyvinyl alcohol, macrogol, and polyethylene oxide;
[18] the process of any one of [15] to [17], wherein the water-soluble polymer is one compound, or two or more compounds selected from the group consisting of hydroxypropyl cellulose, hydroxypropyl methylcellulose, methylcellulose, and hydroxyethyl cellulose;
[19] the process of any one of [15] to [18], wherein an amount of the water-soluble polymer is 1% by weight to 30% by weight with respect to an amount of the methyl methacrylate-butyl methacrylate-dimethylaminoethyl methacrylate copolymer;
[20] the process of any one of [15] to [19], wherein the glidant is one compound, or two or more compounds selected from the group consisting of metal silicates, silicon dioxides, higher fatty acid metal salts, metal oxides, alkaline earth metal salts, and metal hydroxides;
[21] the process of any one of [15] to [20], wherein the glidant is one compound, or two or more compounds selected from the group consisting of talc, kaolin, calcium silicate, magnesium silicate, light anhydrous silicic acid, magnesium stearate, calcium stearate, iron oxide, titanium oxide, calcium carbonate, calcium phosphate, gypsum, magnesium carbonate, aluminum hydroxide, hydrated silicon dioxide, microcrystalline cellulose, synthetic aluminum silicate, heavy anhydrous silicic acid, aluminum magnesium hydroxide, stearic acid, corn starch, magnesium aluminate metasilicate, dibasic calcium phosphate fine granulated, and glyceryl monostearate;
[22] the process of any one of [15] to [21], wherein the glidant is one compound, or two or more compounds selected from the group consisting of talc, kaolin, calcium silicate, magnesium silicate, light anhydrous silicic acid, magnesium stearate, and glyceryl monostearate;
[23] the process of any one of [15] to [22], wherein an amount of the glidant is 1% by weight to 500% by weight with respect to an amount of the methyl methacrylate-butyl methacrylate-dimethylaminoethyl methacrylate copolymer;
[24] the process of any one of [15] to [23], wherein the water-soluble polymer is hydroxypropyl methylcellulose, and an amount of hydroxypropyl methylcellulose is 1% by weight to 30% by weight with respect to an amount of the methyl methacrylate-butyl methacrylate-dimethylaminoethyl methacrylate copolymer;
[25] the process of any one of [15] to [24], wherein the drug is an acidic drug or a salt thereof;
[26] the process of any one of [15] to [25], wherein the drug has an unpleasant taste;
[27] a process of manufacturing the granular pharmaceutical composition for oral administration of any one of [1] to [12], comprising the steps of:
(1) forming a layer comprising a water-soluble insolubilizer and a water-soluble insolubilizing substance outside a drug-containing particle, and
(2) coating the obtained particle with (i) a coating consisting of a methyl methacrylate-butyl methacrylate-dimethylaminoethyl methacrylate copolymer and a water-soluble polymer, or (ii) a coating consisting of a methyl methacrylate-butyl methacrylate-dimethylaminoethyl methacrylate copolymer, a water-soluble polymer, and a glidant;
[28] a process of manufacturing a rapidly disintegrating tablet in the buccal cavity, comprising the step of preparing a medicament using the granular pharmaceutical composition for oral administration of any one of [1] to [12]; and
[29] use of a water-soluble polymer, together with a methyl methacrylate-butyl methacrylate-dimethylaminoethyl methacrylate copolymer, in the manufacture of a granular pharmaceutical composition for oral administration wherein a drug-containing particle is coated to decrease a change in a release rate after compression-molding.

### Advantageous Effects of Invention

According to the present invention, a pharmaceutical formulation having the following effects can be provided:
(1) Drug dosing compliance can be improved by decreasing unpleasantness caused by a drug having an unpleasant taste.
(2) Drug release from the core of the granular pharmaceutical composition after compression-molding can be decreased in a certain period of time when the particles exist in the buccal cavity.
(3) The drug is rapidly released after a certain period of time (released in the upper gastrointestinal tract) to show a sufficient efficacy of the drug.
(4) The present invention can be widely applied to drugs having various properties.

### BRIEF DESCRIPTION OF DRAWINGS

FIG. 1 is a graph showing dissolution profiles of the granular pharmaceutical composition prepared in Example 4 and the compression-molded formulations (rapidly disintegrating tablets in the buccal cavity) containing the same.

FIG. 2 is a graph showing dissolution profiles of the granular pharmaceutical composition prepared in Example 5 and the compression-molded formulations (rapidly disintegrating tablets in the buccal cavity) containing the same.

FIG. 3 is a graph showing dissolution profiles of the granular pharmaceutical composition prepared in Example 6 and the compression-molded formulations (rapidly disintegrating tablets in the buccal cavity) containing the same.

FIG. 4 is a graph showing dissolution profiles of the granular pharmaceutical composition prepared in Example 7 and the compression-molded formulations (rapidly disintegrating tablets in the buccal cavity) containing the same.

FIG. 5 is a graph showing dissolution profiles of the granular pharmaceutical composition prepared in Example 8 and the compression-molded formulations (rapidly disintegrating tablets in the buccal cavity) containing the same.

FIG. 6 is a graph showing dissolution profiles of the granular pharmaceutical composition prepared in Example 9 and the compression-molded formulations (rapidly disintegrating tablets in the buccal cavity) containing the same.

FIG. 7 is a graph showing dissolution profiles of the granular pharmaceutical composition prepared in Example 10 and the compression-molded formulations (rapidly disintegrating tablets in the buccal cavity) containing the same.

FIG. 8 is a graph showing dissolution profiles of the granular pharmaceutical composition prepared in Example 11 and the compression-molded formulations (rapidly disintegrating tablets in the buccal cavity) containing the same.

FIG. 9 is a graph showing dissolution profiles of the granular pharmaceutical composition prepared in Example 12 and the compression-molded formulations (rapidly disintegrating tablets in the buccal cavity) containing the same.

FIG. 10 is a graph showing dissolution profiles of the granular pharmaceutical composition prepared in Example 13 and the compression-molded formulations (rapidly disintegrating tablets in the buccal cavity) containing the same.

FIG. 11 is a graph showing dissolution profiles of the granular pharmaceutical composition prepared in Example 14 and the compression-molded formulations (rapidly disintegrating tablets in the buccal cavity) containing the same.

FIG. 12 is a graph showing dissolution profiles of the granular pharmaceutical composition prepared in Example 16 and the compression-molded formulations (rapidly disintegrating tablets in the buccal cavity) containing the same.

FIG. 13 is a graph showing dissolution profiles of the granular pharmaceutical composition prepared in Example 17 and the compression-molded formulations (rapidly disintegrating tablets in the buccal cavity) containing the same.

FIG. 14 is a graph showing dissolution profiles of the granular pharmaceutical composition prepared in Example 18 and the compression-molded formulations (rapidly disintegrating tablets in the buccal cavity) containing the same.

FIG. 15 is a graph showing dissolution profiles of the granular pharmaceutical composition prepared in Comparative Example 1 and the compression-molded formulations (rapidly disintegrating tablets in the buccal cavity) containing the same.

FIG. 16 is a graph showing dissolution profiles of the granular pharmaceutical composition prepared in Comparative Example 2 and the compression-molded formulations (rapidly disintegrating tablets in the buccal cavity) containing the same.

FIG. 17 is a graph showing dissolution profiles of the granular pharmaceutical composition prepared in Comparative Example 3 and the compression-molded formulations (rapidly disintegrating tablets in the buccal cavity) containing the same.

FIG. 18 is a graph showing dissolution profiles of the granular pharmaceutical compositions prepared in Comparative Example 4.

FIG. 19 is a graph showing the relationship between the content of a water-soluble polymer HPMC in the fifth layer and the f2 function, with respect to the granular pharmaceutical compositions (before tabletting) prepared in Examples 5 and 8 and Comparative Example 1 and the rapidly disintegrating tablets in the buccal cavity (after tabletting under 2 kN) containing the same.

FIG. 20 is a graph showing the relationship between the content of a glidant, talc in the fifth layer and the f2 function, with respect to the granular pharmaceutical compositions (before tabletting) prepared in Examples 5 and 8 and Comparative Example 1 and the rapidly disintegrating tablets in the buccal cavity (after tabletting under 2 kN) containing the same.

### DESCRIPTION OF EMBODIMENTS

Hereinafter the granular pharmaceutical composition for oral administration of the present invention will be explained.

The term “granular pharmaceutical composition” as used herein a drug-containing granular composition which has a size less than a certain value described below and may be orally administered together with one pharmaceutical additive or two or more pharmaceutical additives in various forms. In the case that the shape of the granular composition may be approximate to a sphere, the size of the granular pharmaceutical composition is defined as an average particle size of 2 mm or less. In the case that the shape of the granular pharmaceutical composition is not a sphere, the size of the granular pharmaceutical composition is defined as an average maximum length of 2 mm or less.

In this regard, the lower limit is not particularly limited, so long as it is within a pharmaceutically acceptable range. The size of the granular pharmaceutical composition is, for example, 1 μm or more, 10 μm or more in another embodiment, and 20 μm or more in still another embodiment.

The particle size may be determined by, for example, an optical microscopy method described in the General Tests section of the 15th Edition of the Japanese Pharmacopoeia. In the optical microscopy method, an optical microscopy is used to observe the morphogical appearance and shape of the individual particles either directly with the naked eye or by using a microscopic photograph, in order to measure the particle size. An average length, a triaxial average particle size, or a biaxial average particle size may be used as the particle size.

The “core” is not particularly limited, so long as it is a base which can become a pharmaceutically acceptable particle.

The core is a base which constitutes the granular pharmaceutical composition and is coated with an intermediate layer and a coating used in the present invention. The core is composed of the drug per se, or a pharmaceutically acceptable additive. A particle [for example, crystalline cellulose (particle) (sometimes referred to as microcrystalline cellulose), lactose, starch, or the like] may be used as the core. The drug alone, or a mixture of the drug and a pharmaceutically acceptable additive may be used as the core. A conventional method is used to prepare a particle consisting of one additive, or a particle consisting of two or more additives, which may be used as the core. A particle of one or more additives, which becomes an appropriate core, may be sprayed with a solution or dispersion of the drug and a binder. The size of the core is, for example, 1 μm to 1000 μm, 5 μm to 500 μm in another embodiment, and 10 μm to 200 μm in still another embodiment.

With respect to the term “a change in a release rate after compression-molding”, a change in dissolution properties caused by a certain treatment to a pharmaceutical composition may be evaluated, for example, by using as an index a change in the time (T_{50%}) when 50% of the drug is released from the formulation, as described in Pharmaceutical Development and Technology (volume 8, No. 3, 277-287, 2003). In particular, when the release of a drug having an unpleasant taste is controlled, for example, the time (T_{2%}) when 2% of the drug is released from the formulation may be used as an index of a change in the amount of initial drug dissolution. The “change in a release rate after compression-molding” as used herein may be evaluated by the following two indices DT_{2%}-2 (%) and DT_{50%}-50 (%). Given that the times when the dissolution rate from a pharmaceutical composition before compression-molding is 2% and 50% represent T_{2%} and T_{50%}, respectively, DT_{2%}-2 (%) and DT_{50%}-50 (%) can be calculated from the dissolution rates (after the compression-molding) DT_{2%} and DT_{50%} at the points of T_{2%} and T_{50%}, respectively.

The term “decrease” in the change in a release rate after compression-molding is defined that DT_{2%}-2 (%) is, for example, less than 20%, less than 15% in another embodiment, and less than 10% in still another embodiment. Alternatively, the term “decrease” is defined that DT_{2%}-2 (%) is less than 20%, less than 15% in another embodiment, and less than 10% in still another embodiment, and DT_{50%}-50 (%) is less than 30%, less than 25% in another embodiment, and less than 20% in still another embodiment.

The “drug” used in the present invention is not particularly limited, so long as it is a therapeutically effective active ingredient or a prophylactically effective active ingredient. Examples of the pharmaceutically active ingredient include hypnotic sedatives, sleep-inducing agents, migraine drugs, anti-anxiety drugs, anti-epilepsy drugs, antidepressants, anti-Parkinson's drugs, psychoneurotic drugs, central nervous system drugs, local anesthetics, skeletal muscle relaxants, autonomic nerve drugs, antipyretic analgesic anti-inflammatory agents, antispasmodics, anti-vertigo drugs, cardiotonics, drugs for arrhythmia, diuretics, hypotensives, vasoconstrictors, vasodilators, drugs for the circulatory system, drugs for hyperlipidemia, drugs to promote respiration, antitussives, expectorants, antitussive expectorants, bronchodilators, antidiarrheal agents, drugs for controlling intestinal function, drugs for peptic ulcer, stomachics, antacids, laxatives, cholagogues, gastrointestinal drugs, adrenocortical hormones, hormones, urogenital drugs, vitamins, hemostatics, drugs for liver disease, drugs used for gout, drugs used for diabetes, antihistamines, antibiotics, antibacterials, drugs used against malignant tumors, chemotherapeutic drugs, multisymptom cold medications, nutrition-enhancing health drugs, osteoporosis drugs. and the like.

Examples of the drug include drugs used to treat an overactive bladder, such as solifenacin and tolterodine, sleep-inducing drugs, such as diphenhydramine and lorazepam, anti-inflammatory, antipyretic antispasmodics or analgesics, such as indomethacin, diclofenac, diclofenac sodium, codeine, ibuprofen, phenylbutazone, oxyfenbutazone, mepirizole, aspirin, etenzamide, acetaminophen, aminopyrine, phenacetin, butyl scopolamine bromide, morphine, etomidoline, pentazocine, fenoprofen calcium, naproxen, celecoxib, vardecoxib, tramadole, migraine drugs, such as sumatriptan, anti-rheumatic drugs, such as etodolac, anti-tuberculosis drugs, such as isoniazide, ethambutol chloride, drugs for the circulatory system, such as isosorbid nitrate, nitroglycerin, nifedipine, bardnidipine hydrochloride, nicardipine hydrochloride, dipyridamole, anrinone, indenolol hydrochloride, hydralazine hydrochloride, methyldopa, fuirosemide, spironolactone, guanetidine nitrate, resperine, amosulalol hydrochloride, lisinopril, methoprolol, pilocarpine, telmisartan, psychoneurotic drugs, such as chlorpromazine hydrochloride, amitriptyline hydrochloride, nemonapride, haloperidol, moperone hydrochloride, perphenazine, diazepam, lorazepam, chlordiazepoxide, adinazolam, alprazolam, methylphenidate, milnasivran, peroxetin, risperidone, sodium valproate, antidepressants, such as imipramine, antiemetics, such as methoclopramide, ramosetron hydrochloride, granisetron hydrochloride, ondansetron hydrochloride, azasetron hydrochloride, antihistamines, such as chlorpheniramine maleate, vitamins, such as thiamine nitrate, tocopherol acetate, sicotiamine, pyridoxal phosphate, cobamamide, ascorbic acid, nicotinamide, antigout drugs, such as allopurinol, colchicine, probenamide, anti-Parkinson's drugs, such as levodopa, selegiline, etc., hypnotic sedatives, such as amobarbital, bromwarelyl urea, midazolam, chloral hydrate, etc., anti-malignant tumor drugs, such as fluorouracil, carmofuir, aclarubicin hydrochloride, cyclophosphamide, thiotepa, anti-allergy drugs, such as pseudoephedrine, terfenadine, decongestants, such as phenyl propanolamine, ephedrines, drugs used to treat diabetes, such acethexamide, insulin, torbutamide, desmopressine, glibizide, and nateglinide, diuretics, such as hydrochlorthiazide, polythiazide, triaterene, bronchodilators, such as aminophylline, formoterol fumarate, theophylline, antitussives, such as codeine phosphate, noscapine, dimemorphan phosphate, dextromethorphan, antiarrythmia drugs, such as quinidine nitrate, digitoxin, propafenone hydrochloride, procainamide, surface anesthetics, such as aminoethyl benzoate, lidocaine, dibucaine hydrochloride, antiepilepsy drugs, such as phenytoin, etosuccimide, primidone, synthetic corticosteroids, such as hydrocortisone, prednisolone, triamcinolone, betamethasone, drugs for the digestive tract, such as famotidine, ranitidine hydrochloride, cimetidine, sucralfate, sulpiride, tepronone, praunotol, 5-aminosalicylic acid, sulfasalazine, omeprazole, lansoprazole, drugs for the central nervous system, such as indeloxazine, idebenone, thiapride hydrochloride, bifermerane hydrochloride, calcium homopanthothenate, agents for treatment of hyperlipidemia, such as pravastatin sodium, sinvastatin, lovastatin, atorvastatin, antibiotics, such as ampicillin phthalizyl hydrochloride, cefotetan, josamycin, BPH therapeutic agents, such as tamsulosin, doxazocin mesilate, terazosine hydrochloride, anti-asthma drugs, such as pranlukast, zafirlukast, albuterol, ambroxole, budesonide, leverbuterol, prostaglandin I2 derivative agents for improving peripheral circulation, such as velaprost sodium, agents for treatment of various complications of diabetes, agents for treatment of skin ulcers, and the like.

These drugs may be used in a free form, or as a pharmaceutically acceptable salt. These drugs may be used alone, or a combination of two or more thereof.

As the drug used in the present invention, a drug which requires timed release and further requires rapid release after lag time, in particular, a drug having an unpleasant taste (such as a bitter taste or an astringent taste), or a drug which may raise possible problems such as adverse events or increased individual differences in pharmacological effects accompanied by the absorption thereof in the buccal cavity, may be used. The drug having an unpleasant taste is not particularly limited, and the drugs described in International Publication No. WO 02/02083 may be used (An embodiment in which drugs excluding atorvastatin and pharmaceutically acceptable salts thereof are used is included in the present invention.).

In the present invention, the lower the water-solubility of the drug is, more efficiently the desired effects of the present invention can be obtained. The solubility of the drug is not particularly limited, so long as it is pharmaceutically acceptable. The solubility is, for example, 500 μg/mL or less in a test liquid of pH 1.2, 200 μg/mL or less in another embodiment, and 50 μg/mL or less in still another embodiment. The form of the drug used in the present invention is not particularly limited, so long as it is pharmaceutically acceptable. For example, an acidic drug or a salt thereof, or atorvastatin or a pharmaceutically acceptable salt thereof in another embodiment, may be used.

Atorvastatin or pharmaceutically acceptable salts thereof which may be used in the present invention include atorvastatin calcium hydrate disclosed in U.S. Pat. No. 5,273,995 having a chemical name of [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate. Atorvastatin calcium hydrate has a structure of the formula:

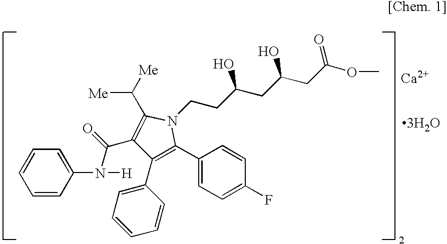

and is placed on the market as Lipitor (registered trademark).

Atorvastatin or a pharmaceutically acceptable salt thereof is a selective and competitive inhibitor of HMG-CoA reductase. Examples of the pharmaceutically acceptable salt include a metal salt such as an alkaline metal salt and an alkaline earth metal salt, and an amine salt such as an organic amine. In another embodiment, examples thereof include salts with sodium, potassium, lithium, calcium, magnesium, aluminum, iron, zinc, calcium carbonate, calcium hydroxide, magnesium carbonate, magnesium hydroxide, magnesium silicate, magnesium aluminate, or aluminum magnesium hydroxide. In still another embodiment, a calcium salt may be used. Atorvastatin calcium is a potent lipid-lowering compound, and thus, is useful as a lipid-lowering medication and/or a cholesterol-lowering medication, and also useful in treating osteoporosis, benign prostatic hypertrophy (BPH), and Alzheimer disease. Examples of crystalline-form atorvastatins include types I, II, IV, V, VI, VII, VIII, IX, X, XI, XII, XIII, XIV, XV, XVI, XVII, XVIII, and XIX and, in another embodiment, type I. The term “type I crystal” as used herein means crystalline Form I atorvastatin hydrate disclosed in Japanese Patent No. 3296564.

The content of the drug is not particularly limited, so long as it is a pharmaceutically effective amount for prevention or treatment. The drug is administered to an adult patient at a daily dose of 10 ng to 5000 mg in general, a daily dose of 500 μg to 1000 mg in another embodiment, a daily dose of 1 mg to 100 mg in still another embodiment. The content of the drug may be generally selected in accordance with the type of the drug, the application (indications) of the drug, or the age (or the weight) of a patient, and is not particularly limited, so long as it is a therapeutically or prophylactically effective amount. For example, the content is 0.0001% by weight to 90% by weight with respect to the amount of the “granular pharmaceutical composition” or the pharmaceutical formulation of the present invention, 0.0001% by weight to 80% by weight in another embodiment, and 0.5% by weight to 70% by weight in still another embodiment.

When the drug is atorvastatin or a pharmaceutically acceptable salt thereof, the content thereof is not particularly limited, so long as it is a pharmaceutically effective amount for prevention or treatment. The daily dose is approximately 2.5 mg to approximately 80 mg, approximately 5 mg to approximately 500 mg in another embodiment, and approximately 2.5 mg to approximately 80 mg in still another embodiment. Alternatively, the drug is administered to an adult patient at a daily dose of approximately 0.1 mg/kg to approximately 8.0 mg/kg. A daily dose in another embodiment is within a range of approximately 0.1 mg/kg to approximately 2.0 mg/kg. The content may be changed or adjusted to 5 mg to 80 mg, or 5 mg to 100 mg in another embodiment, in accordance with the effect or application. In common treatment, a drug is administered to a patient in an amount less than the optimum dose at an early stage. The dose is gradually increased in accordance with the conditions until optimum effect is achieved. The daily dose can be divided into multiple doses per day, if necessary.

The content of the drug may be generally selected in accordance with the type of the drug, the application (indications) of the drug, or the age (or the weight) of a patient, and is not particularly limited, so long as it is a therapeutically or prophylactically effective amount. For example, the content is 0.5% by weight to 90% by weight with respect to the amount of the “granular pharmaceutical composition” or the pharmaceutical formulation of the present invention, 0.5% by weight to 80% by weight in another embodiment, and 0.5% by weight to 70% by weight in still another embodiment.

The “methyl methacrylate-butyl methacrylate-dimethylaminoethyl methacrylate copolymer” [hereinafter sometimes referred to as aminoalkylmethacrylate copolymer E, copolymer E, Eudragit (registered trademark) E (Evonik Degussa GmbH), methyl methacrylate butyl methacrylate 2-dimethylaminoethyl methacrylate copolymer, or the like] used in the present invention is a polymer which is commercially available as the product name of Eudragit (registered trademark) E100 (Evonik Degussa GmbH) or Eudragit (registered trademark) EPO (Evonik Degussa GmbH) and has an average molecular weight of 150,000 [Iyakuhin Tenkabutsu Kikaku (Japanese Pharmaceutical Excipients), P 76-77, 1998, Yakuji Nippo Limited; and Handbook of Pharmaceutical Excipients second edition p 362-366, 1994, American Pharmaceutical Association, Washington and The Pharmaceutical Press, London].

The content of the methyl methacrylate-butyl methacrylate-dimethylaminoethyl methacrylate copolymer, with respect to the amount of the drug-containing particles, may be, for example, 1% by weight to 500% by weight, 5% by weight to 300% by weight in another embodiment, and 10% by weight to 150% by weight in still another embodiment. The content thereof, with respect to the amount of the particles coated with the intermediate layer, may be, for example, 1% by weight to 300% by weight, 5% by weight to 200% by weight in another embodiment, and 5% by weight to 150% by weight in still another embodiment. The content of the coating contained in the granular pharmaceutical composition may be, for example, 1% by weight to 200% by weight, 5% by weight to 100% by weight in another embodiment, and 5% by weight to 50% by weight in still another embodiment.

The “water-soluble polymer” used in the present invention is not particularly limited, so long as it is pharmaceutically acceptable. Further, the water-soluble polymer is not particularly limited, so long as it can constitute the coating component together with copolymer E, and has a function capable of decreasing the dissolution of the drug from the granular pharmaceutical composition after compression-molding, by coating the granular pharmaceutical composition with the coating.

Examples of the water-soluble polymer include powdered acacia, sodium alginate, gelatinized starch, casein sodium, carrageenan, a carboxyvinyl polymer, carboxymethyl starch sodium, carmellose sodium, xanthan gum, dextran, dextrin, hydroxypropyl cellulose, hydroxypropyl methylcellulose, methylcellulose, hydroxyethyl cellulose, pullulan, povidone, copolyvidone, polyoxyethylene-polyoxypropylene glycol, polyvinyl acetal diethyl aminoacetate, a polyvinyl alcohol-polyethylene glycol graft copolymer, polyvinyl alcohol, macrogol, and polyethylene oxide. In another embodiment, hydroxypropyl cellulose, hydroxypropyl methylcellulose, methylcellulose, hydroxyethyl cellulose, povidone, copolyvidone, a polyvinyl alcohol-polyethylene glycol graft copolymer, polyvinyl alcohol, macrogol, polyethylene oxide, or the like may be used; in still another embodiment, hydroxypropyl cellulose, hydroxypropyl methylcellulose, methylcellulose, hydroxyethyl cellulose, or the like may be used; and in still another embodiment, hydroxypropyl cellulose, hydroxypropyl methylcellulose, hydroxyethyl cellulose, or the like may be used. As hydroxypropyl methylcellulose, a polymer which is commercially available as the product name of hypromellose, Japanese Pharmacopoeia (Shin-Etsu Chemical Co., Ltd.)(viscosity indicated=3 mPa·s to 15 mPa·s) may be used.

These water-soluble polymers may be used alone, or as an appropriate combination of two or more thereof.

The composition ratio (mixing ratio) of the water-soluble polymer to the methyl methacrylate-butyl methacrylate-dimethylaminoethyl methacrylate copolymer (copolymer E), which are used in the present invention, is appropriately selected in accordance with, in general, properties, stability, or absorption site of the drug, or the type or application of the formulation. The content of the water-soluble polymer with respect to copolymer E is, for example, 1% by weight to 30% by weight, 5% by weight to 20% by weight in another embodiment, and 5% by weight to 15% by weight in still another embodiment.

The coating amount of the coating containing copolymer E and the water-soluble polymer in the present invention is appropriately selected in an appropriate ratio. The content of the coating with respect to the drug-containing core is, for example, 1% by weight to 500% by weight, 5% by weight to 300% by weight in another embodiment, and 10% by weight to 150% by weight in still another embodiment. When the coating amount is less than 1% by weight, the surface of the granular pharmaceutical composition cannot be uniformly coated and the thickness of the coating layer is very thin, and thus, it is anticipated that a change in the dissolution rate of the drug from the granular pharmaceutical composition, the change being caused by compression-molding, will increased.

The content of the coating with respect to the particles coated with the intermediate layer is, for example, 1% by weight to 500% by weight, 5% by weight to 200% by weight in another embodiment, and 10% by weight to 100% by weight in still another embodiment. The content of the coating contained in the granular pharmaceutical composition is, for example, 1% by weight to 200% by weight, 5% by weight to 100% by weight in another embodiment, and 5% by weight to 50% by weight in still another embodiment.

An embodiment of the granular pharmaceutical composition of the present invention may contain the “glidant”, if desired. The formulation of the glidant is not limited to a specific process. For example, when the granular pharmaceutical composition is manufactured by a fluidized bed granulation method, mixing of components or drying of particles causes static electricity, which sometimes interferes with the fluidization. The glidant is not particularly limited, so long as it has a function, for example, capable of neutralizing the generated static electricity, and can improved the fluidization in a coating step. Examples of the glidant include metal silicates, silicon dioxides, higher fatty acid metal salts, metal oxides, alkaline earth metal salts, and metal hydroxides. In another embodiment, talc, kaolin, calcium silicate, magnesium silicate, light anhydrous silicic acid, magnesium stearate, calcium stearate, iron oxide, titanium oxide, calcium carbonate, calcium phosphate, gypsum, magnesium carbonate, aluminum hydroxide, hydrated silicon dioxide, microcrystalline cellulose, synthetic aluminum silicate, heavy anhydrous silicic acid, aluminum magnesium hydroxide, stearic acid, corn starch, magnesium aluminate metasilicate, dibasic calcium phosphate fine granulated, or glyceryl monostearate may be used. In still another embodiment, talc, kaolin, calcium silicate, magnesium silicate, light anhydrous silicic acid, magnesium stearate, or glyceryl monostearate may be used. These glidants may be added alone, or as an appropriate combination of two or more thereof.

The content of the glidant with respect to the drug-containing particles is, for example, 1% by weight to 500% by weight, 1% by weight to 200% by weight in another embodiment, and 5% by weight to 100% by weight in still another embodiment. The content of the glidant with respect to copolymer E is, for example, 1% by weight to 200% by weight, 5% by weight to 100% by weight in another embodiment, and 20% by weight to 60% by weight in still another embodiment.

The granular pharmaceutical composition for oral administration of the present invention may be formulated appropriately using one or more various pharmaceutical fillers if desired. Such fillers are not particularly limited, so long as they are pharmaceutically acceptable and pharmacologically acceptable, and include, for example, binders, disintegrating agents, acidulants, foaming agents, artificial sweeteners, flavors, lubricants, coloring agents, stabilizers, buffers, antioxidants, surfactants, and the like.

Examples of the binders include hydroxypropyl methylcellulose, powdered acacia, and the like.

Examples of the disintegrating agents include corn starch, potato starch, carmellose calcium, carmellose sodium, and the like.

Examples of the acidulants include citric acid, tartaric acid, malic acid, and the like.

Examples of the foaming agents include sodium bicarbonate and the like.

Examples of the artificial sweeteners include saccharin sodium, dipotassium glycyrrhizinate, aspartame, stevia, somatin, and the like.

Examples of the flavors include lemon, lemon lime, orange, menthol, and the like.

Examples of the lubricants include magnesium stearate, calcium stearate, sucrose fatty acid ester, polyethylene glycol, talc, stearic acid, and the like.

Examples of the coloring agents include yellow ferric oxide, red ferric oxide, food yellow No. 4, food yellow No. 5, food red No. 3, food red No. 102, food blue No. 3, and the like.

Examples of the buffers include citric acid, succinic acid, fumaric acid, tartaric acid, ascorbic acid, and salts thereof; glutamic acid, glutamine, glycine, aspartic acid, alanine, arginine, and salts thereof; magnesium oxide, zinc oxide, magnesium hydroxide, phosphoric acid, boric acid, and salts thereof; and the like.

Examples of the antioxidants include ascorbic acid, dibutyl hydroxytoluene, propyl gallate, and the like.

Examples of the surfactants include polysorbate 80, sodium laurylsulfate, polyoxyethylene hydrogenated castor oil, and the like.

These pharmaceutical fillers may be appropriately added alone, or as a combination of two or more thereof, in an appropriate amount.

The content of these pharmaceutical fillers with respect to the drug-containing particles is, for example, 1% by weight to 100% by weight, 5% by weight to 80% by weight in another embodiment, and 10% by weight to 50% by weight in still another embodiment.

In the granular pharmaceutical composition for oral administration of the present invention, the coating containing the methyl methacrylate-butyl methacrylate-dimethylaminoethyl methacrylate copolymer and the water-soluble polymer may be used to directly coat the drug-containing core, or to coat particles prepared by previously coating the drug-containing core with one layer or two or more layers. In the case of coating after coating the core with one layer or two or more layers, the “intermediate layer” may be arranged between the drug-containing core and the coating containing the methyl methacrylate-butyl methacrylate-dimethylaminoethyl methacrylate copolymer and the water-soluble polymer. The “intermediate layer” means a coating layer containing one, or two or more water-soluble insolubilizer and one, or two or more water-soluble insolubilizing substance. The drug-containing core may be directly coated with the intermediate layer. Alternatively, the drug-containing core may be previously coated with one or more components which do not avoid lag time generation and subsequent rapid drug release, as one coating layer or two or more coating layers, and then, coated with the intermediate layer. The intermediate layer contains two or more essential components (the insolubilizer and the insolubilizing substance), which may be contained in a single layer and uniformly or unevenly distributed in the single layer. Alternatively, the two or more essential components (the insolubilizer and the insolubilizing substance) contained in the intermediate layer may be partitioned into two or more plural layers. In this case, plural essential components may be partitioned in any combination and in any arrangement. When the coating layers containing plural essential components are plural layers, such plural coating layers are collectively designated the intermediate layer.

The coating amount of the intermediate layer, with respect to the drug-containing particles, is, for example, 1% by weight to 500% by weight, 1% by weight to 300% by weight in another embodiment, and 20% by weight to 200% by weight in still another embodiment. The content of the intermediate layer with respect to the total weight of the granular composition is, for example, 0.1% by weight to 95% by weight, 1% by weight to 85% by weight in another embodiment, and 3% by weight to 80% by weight in still another embodiment.

As an embodiment of the granular pharmaceutical composition of the present invention, the outside of the coating layer containing the methyl methacrylate-butyl methacrylate-dimethylaminoethyl methacrylate copolymer and the water-soluble polymer may be further coated with the above-mentioned pharmaceutical fillers. Examples of such additives for further coating include amino acids such as glycine and alanine, sweeteners such as glycyrrhizinic acid, sugars such as sucrose, fructose, maltose, glucose, and cyclodextrin, sugar alcohols such as mannitol, xylitol, maltitol, and sorbitol, and the like. The coating layer (outer layer) containing the pharmaceutical fillers may appropriately contain one pharmaceutical filler or two or more pharmaceutical fillers, in an appropriate amount.

The coating amount of the outer layer, with respect to the drug-containing particles, is, for example, 1% by weight to 200% by weight, 1% by weight to 100% by weight in another embodiment, and 5% by weight to 40% by weight in still another embodiment. The content of the outer layer with respect to the total weight of the granular composition is, for example, 1% by weight to 50% by weight, 1% by weight to 25% by weight in another embodiment, and 5% by weight to 10% by weight in still another embodiment.

The granular pharmaceutical composition of the present invention may be used to prepare various pharmaceutical formulations, which include, for example, powder, granules, dry syrups, tablets, rapidly disintegrating tablets in the buccal cavity, and the like.

Hereinafter the rapidly disintegrating tablet in the buccal cavity of the present invention containing the granular pharmaceutical composition of the present invention will be explained, but the pharmaceutical formulation of the present invention is not limited thereto.

The term “rapidly disintegrating tablet in the buccal cavity” as used herein means a tablet (or other formulations similar to a tablet) which is disintegrated in the buccal cavity within 2 minutes, 1 minute in another embodiment, and 45 seconds in still another embodiment, by substantially saliva only, without taking water for swallowing the tablets.

The granular pharmaceutical composition of the present invention may be contained in a rapidly disintegrating tablet in the buccal cavity. The granular pharmaceutical composition of the present invention may be used as a drug contained in known rapidly disintegrating tablets in the buccal cavity, as described in WO 95/20380 (corresponding U.S. Pat. No. 5,576,014), WO 2002/92057 (corresponding US Patent Application Publication No. 2003/099701), U.S. Pat. No. 4,305,502, U.S. Pat. No. 4,371,516, Japanese Patent No. 2807346 (corresponding U.S. Pat. No. 5,466,464), Japanese Unexamined Patent Publication (kokai) No. 5-271054 (corresponding EP Patent No. 553777), Japanese Unexamined Patent Publication (kokai) No. 10-182436 (corresponding U.S. Pat. No. 5,958,453), Japanese Patent No. 3412694 (corresponding U.S. Pat. No. 5,223,264), WO 98/02185 (corresponding U.S. Pat. No. 6,287,596), and WO 2008/032767 (corresponding US Patent Application Publication No. 2008/0085309), and a base for rapidly disintegrating tablets in the buccal cavity, as described in these documents, may be used to prepare a rapidly disintegrating tablet in the buccal cavity in accordance with a method as described in these documents. As described above, examples of rapidly disintegrating tablets in the buccal cavity containing a granular pharmaceutical composition include rapidly disintegrating tablets in the buccal cavity described in Japanese Patent No. 3412694 (corresponding U.S. Pat. No. 5,223,264) and Japanese Unexamined Patent Publication (kokai) No. 2003-55197, and the granular pharmaceutical composition of the present invention may be contained in these rapidly disintegrating tablets in the buccal cavity.

In general, rapidly disintegrating tablets in the buccal cavity are mainly classified into a molding type, a wet type, and a conventional tabletting type. The granular pharmaceutical composition of the present invention may be contained in any type of these rapidly disintegrating tablets in the buccal cavity. The molding type is the one prepared by filling a solution or suspension containing a filler or the like in a mold, and drying it, as disclosed in, for example, Japanese Patent No. 2807346 (corresponding to U.S. Pat. No. 5,466,464). The molding type of rapidly disintegrating tablet in the buccal cavity containing the granular pharmaceutical composition of the present invention may be prepared, for example, by filling a solution or suspension containing the granular pharmaceutical composition of the present invention, a filler such as a saccharide, and a binder such as gelatin or agar into a PTP pocket, and removing water therefrom by lyophilization, drying under reduced pressure, low-temperature drying, or the like. The wet type is the one prepared by moistening a filler such as a saccharide, tabletting it at a low pressure, and drying the tablet, as disclosed in, for example, Japanese Patent No. 3069458 (corresponding to U.S. Pat. No. 5,501,861 and U.S. Pat. No. 5,720,974). The wet type may be prepared, for example, by moistening the granular pharmaceutical composition of the present invention and a filler such as a saccharide with a small amount of water or a mixture of water and alcohol, tabletting the wet mixture at a low pressure, and drying it.

The conventional tabletting type is the one prepared by carrying out a conventional tabletting step, as disclosed in WO 95/20380 (corresponding to U.S. Pat. No. 5,576,014), WO 2002/92057 (corresponding US Patent Application Publication No. 2003/099701), Japanese Unexamined Patent Publication (kokai) No. 10-182436 (corresponding to U.S. Pat. No. 5,958,453), Japanese Unexamined Patent Publication (kokai) No. 9-48726, Japanese Unexamined Patent Publication (kokai) No. 8-19589 (corresponding to U.S. Pat. No. 5,672,364), Japanese Patent No. 2919771, Japanese Patent No. 3069458 (corresponding to U.S. Pat. No. 5,501,861, and corresponding to U.S. Pat. No. 5,720,974), and WO 2008/032767 (corresponding US Patent Application Publication No. 2008/0085309). The conventional tabletting type of rapidly disintegrating tablet in the buccal cavity containing the granular pharmaceutical composition of the present invention may be prepared by granulating the granular pharmaceutical composition of the present invention and a filler such as a saccharide having a low moldability with a solution or suspension containing a saccharide having a high moldability or a water-soluble polymer, and compression-molding granules into a compression-molded product, and optionally further drying the compression-molded product under a humidity condition, as disclosed in, for example, WO 95/20380 (corresponding to U.S. Pat. No. 5,576,014) and Japanese Patent No. 2919771. A conventional tabletting type of rapidly disintegrating tablet in the buccal cavity as disclosed in WO 99/47124 (corresponding to U.S. Pat. No. 6,589,554) may be prepared, for example, by compression-molding the granular pharmaceutical composition of the present invention and a filler such as a crystalline saccharide using an amorphous saccharide, and drying it under a humidity condition. A conventional tabletting type of rapidly disintegrating tablet in the buccal cavity as disclosed in WO 2002/92057 (corresponding US Patent Application Publication No. 2003/099701) may be prepared, for example, by compression-molding a mixture of the granular pharmaceutical composition of the present invention and a filler with a saccharide having a melting point lower than that of the filler, and heating the compression-molded product to form a crosslinkage with a melted and solidified saccharide having a low melting point. These treatments such as drying under a humidity condition or heating can improve the tablet strength of the rapidly disintegrating tablet in the buccal cavity. A conventional tabletting type of rapidly disintegrating tablet in the buccal cavity as disclosed in WO 2008/032767 (corresponding US Patent Application Publication No. 2008/0085309) may be prepared, for example, by compression-molding a mixture of the granular pharmaceutical composition of the present invention and a filler with a treated starch having a degree of gelatinization of 30% to 60%.

As fillers used in the rapidly disintegrating tablet in the buccal cavity of the present invention, conventional fillers may be used, and pharmaceutically acceptable saccharides are preferable. More particularly, a saccharide having a low moldability may be used with respect to techniques utilizing moldability of saccharides, a crystalline saccharide may be used with respect to techniques for improving a tablet strength by crystalline/amorphous properties of saccharides and drying under a humidity condition, and a saccharide having a high melting point as well as conventional fillers may be used with respect to a crosslinking technique using a melted and solidified saccharide.

The term “saccharide having a low moldability” as used herein means that, for example, when 150 mg of saccharide is formed into tablets using a punch of 8 mm in diameter under a tabletting pressure of 10 kg/cm² to 50 kg/cm², the tablets show a hardness of 0 kp to 2 kp. The term “saccharide having a high moldability” as used herein means that the tablets show a hardness of 2 kp or more, under the same conditions. Examples of saccharides having a low moldability, which are pharmaceutically acceptable, include lactose, mannitol, glucose, sucrose, xylitol, and erythritol. These saccharides may be used alone, or as an appropriate combination of two or more thereof. Examples of saccharides having a high moldability, which are pharmaceutically acceptable, include maltose, maltitol, sorbitol, and trehalose. These saccharides also may be used alone, or as an appropriate combination of two or more thereof.

Examples of the “crystalline saccharide”, which is pharmaceutically acceptable, include mannitol, maltitol, erythritol, and xylitol. These saccharides may be used alone, or as an appropriate combination of two or more thereof. Examples of the “amorphous saccharide”, which is pharmaceutically acceptable, include lactose, sucrose, glucose, sorbitol, maltose, and trehalose. These saccharides also may be used alone, or as an appropriate combination of two or more thereof.

Examples of the “saccharide having a high melting point”, which is pharmaceutically acceptable, include xylitol, trehalose, maltose, sorbitol, erythritol, glucose, sucrose, maltitol, and mannitol. These saccharides may be used alone, or as an appropriate combination of two or more thereof. Examples of the “saccharide having a low melting point”, which is pharmaceutically acceptable, include xylitol, trehalose, maltose, sorbitol, erythritol, glucose, sucrose, maltitol, and mannitol. These saccharides also may be used alone, or as an appropriate combination of two or more thereof. Examples of a binder for rapidly disintegrating tablets in the buccal cavity include maltitol and copolyvidone. These binders may be used alone, or as an appropriate combination of two or more thereof.

When a water-soluble polymer is used instead of the saccharide having a high moldability, for example, hydroxypropyl cellulose, hydroxypropylmethyl cellulose, povidone, polyvinyl alcohol, powdered acacia, gelatin, pullulan, and the like are preferable.

The term “gelatinization” as used herein means a swelling caused by physically treating a starch, thereby introducing water into the space between starch molecules. An increasing degree of gelatinization means the progress of gelatinization. Examples of a treated starch include corn starch, wheat starch, potato starch, rice starch, and cassava starch.

The content of fillers used in the rapidly disintegrating tablet in the buccal cavity containing the granular pharmaceutical composition of the present invention may be appropriately adjusted in accordance with the content of the granular pharmaceutical composition of the present invention, the size of the tablet, and/or the like, and it is preferably 20 mg to 1000 mg per tablet in general, 50 mg to 900 mg in another embodiment, and 100 mg to 800 mg in still another embodiment.

The contents of the saccharide having a high moldability, the water-soluble polymer, the amorphous saccharide, and the saccharide having a low melting point vary according to each technique, but it is preferably 0.5% by weight to 40% by weight with respect to the weight of the filler(s), 2% by weight to 30% by weight in another embodiment, and 5% by weight to 20% by weight in still another embodiment, or it is preferably 1% by weight to 20% by weight with respect to the weight of the formulation.

With respect to the type, formulation, content, and the like of other additives optionally used, the contents of the above-mentioned patent references disclosing rapidly disintegrating tablets in the buccal cavity are incorporated herein by reference.

In the case that the granular pharmaceutical composition of the present invention is contained in the rapidly disintegrating tablet in the buccal cavity, the granular pharmaceutical composition may be contained therein in an amount corresponding to 0.5% by weight to 90% by weight with respect to that of the rapidly disintegrating tablet in the buccal cavity, preferably 1% by weight to 80% by weight, and 5% by weight to 60% by weight in another embodiment.

Hereinafter the process of manufacturing the granular pharmaceutical composition of the present invention will be explained, but the present invention is not limited thereto.

The granular pharmaceutical composition of the present invention may be produced in accordance with a known method per se, such as coating, drying, heating, tabletting, or the like.

To obtain the granular pharmaceutical composition of the present invention, the drug-containing core is coated with the coating used in the present invention. A particle consisting only of the drug may be used as the drug-containing core. Alternatively, a conventional technique may be used to prepare a particle consisting of the drug and an additive or two or more additives, in order to use as the drug-containing core. In this process of preparing the particle consisting of the drug and additive(s), for example, the drug and one or more appropriate fillers (such as crystalline cellulose, lactose, corn starch, or the like) may be mixed, together with a binder (such as hydroxypropyl cellulose or the like) if necessary, and granulated, sieved, and dried. Alternatively, an additive particle [for example, crystalline cellulose (particle) (sometimes referred to as microcrystalline cellulose), purified sucrose spheres, sucrose-starch spheres, or the like] as an appropriate core may be sprayed with a solution or dispersion liquid containing the drug and a binder.

As a method of coating the outside of the drug-containing with the coating used in the present invention, any method capable of coating a granular pharmaceutical composition, such as a fluidized bed coating apparatus, a tumbling coating apparatus, a centrifugal tumbling coating apparatus, or the like, may be used. For example, in a fluidized bed granulating and side-spray coating apparatus with a side-spray, an appropriate amount of liquid containing a coating component may be sprayed using a spray gun, while the drug-containing core is fluidized by a warm air. The liquid containing a coating component may be prepared by dissolving or dispersing the essential component in a solvent such as water, ethanol, or methanol. These solvents may be used as an appropriate mixture.

After the outside of the drug-containing core is coated with the intermediate layer, or after the granular pharmaceutical composition of the present invention is coated with the pharmaceutical filler, the resulting particles may be coated with the coating used in the present invention.

A preferred spray rate for coating may vary according to a process or a scale thereof. At a 1 kg scale production by the fluidized bed granulating, a preferred spray rate is, for example, 2 g/min to 8 g/min, and 5 g/min to 7 g/min in another embodiment.

A preferred temperature of the product when the drug-containing core is coated with the intermediate layer or the layer capable of controlling the amount of water penetration is 15° C. to 60° C., and 15° C. to 45° C. in another embodiment.

The granular pharmaceutical composition obtained by applying the drug-containing particles with the coating may be further dried, heated, or the like.

The particle size of the granular pharmaceutical composition of the present invention is not particularly limited, so long as the maximum length thereof is 2 mm or less. In the case that the granular pharmaceutical composition is contained in the rapidly disintegrating tablet in the buccal cavity, it is not particularly limited, so long as graininess like sands is not unpleasant in the administration, and the average particle size is preferably 350 μm or less, 1 μm to 350 μm in another embodiment, and 20 μm to 350 μm in still another embodiment.

Examples of the tabletting include a direct tabletting method in which drug-containing particles are mixed with an appropriate additive(s), and the mixture is compression-molded to obtain tablets; a method in which a composition obtained by a wet granulation (the granulation is carried out by spraying a mixture of drug-containing particles and additives with a binder liquid) or a melting granulation (the granulation is carried out by heating a mixture of drug-containing particles and an appropriate low-melting substance) is formed into tablets; and the like.

A rotary tabletting machine, a single punch tabletting machine, and the like may be used as a tabletting machine. A method as well as an apparatus is not particularly limited, so long as a compression-molded product can be pharmaceutically produced.

The use of the methyl methacrylate-butyl methacrylate-dimethylaminoethyl methacrylate copolymer and the water-soluble polymer of the present invention is a use of the methyl methacrylate-butyl methacrylate-dimethylaminoethyl methacrylate copolymer and the water-soluble polymer in the manufacture of a granular pharmaceutical composition for oral administration in which a core containing a drug having a bitter taste is coated to decrease a change in a release rate after compression-molding. The explanation for the granular pharmaceutical composition of the present invention is cited as the detail description of the invention for the use.

Hereinafter a process of manufacturing the rapidly disintegrating tablet in the buccal cavity containing the granular pharmaceutical composition of the present invention will be explained.

As an example, the following example is the rapidly disintegrating tablet in the buccal cavity described in International Publication No. WO 95/20380 (corresponding to U.S. Pat. No. 5,576,014). The tablet can be manufactured by the steps of mixing the granular pharmaceutical composition of the present invention with the saccharide having a low moldability; coating and/or granulating this mixture by spraying the saccharide having a high moldability as a binder; and compression-molding the resulting granulated product. This prepared molded product may be further treated by humidifying and drying to increase the hardness thereof.

The “humidifying” is appropriately determined in accordance with the apparent critical relative humidity of the saccharides contained therein, and is generally carried out at the critical relative humidity or higher. The humidity is, for example, 30% RH to 100% RH, and 50% RH to 90% RH in another embodiment. In this case, the temperature is preferably 15° C. to 50° C., and 20° C. to 40° C. in another embodiment. The treatment time is 1 hour to 36 hours, and 12 hours to 24 hours in another embodiment.

The “drying” is not particularly limited, so long as it is a step in which the moisture absorbed by humidifying is removed. The drying temperature may be, for example, 10° C. to 100° C., 20° C. to 60° C. in another embodiment, and 25° C. to 40° C. in still another embodiment. The treatment time is 0.5 hour to 6 hours, and 1 hour to 4 hours in another embodiment.

As another example, the following example is the rapidly disintegrating tablet in the buccal cavity described in International Publication No. WO 2002/92057 (corresponding to U.S. Patent Application Publication No. 2003/099701). The tablet can be manufactured by the steps of mixing the granular pharmaceutical composition of the present invention, a filler having a high melting point, and a saccharide having a low melting point; coating and/or granulating this mixture by spraying a binder for a rapidly disintegrating tablet in the buccal cavity; and compression-molding the resulting granulated product. When the filler having a high melting point is combined with the saccharide having a low melting point, the heating step may be used to increase the hardness of the resulting molded product. The “heating” is determined in accordance with the melting point of the saccharide having a low melting point contained therein, and is generally carried out at a temperature the same as or higher than the melting point of the saccharide having a low melting point, and lower than the melting point of the filler having a high melting point. The treatment time is 0.5 minute to 120 minutes, and 1 minute to 60 minutes in another embodiment.

### Examples

The present invention will be further illustrated by, but is by no means limited to, the following Examples. The numbers which represent each layer in the following Examples do not limit the present invention.

### Example 1

As atorvastatin calcium trihydrate, crystalline Form I atorvastatin prepared in accordance with the method described in Examples of Japanese Patent No. 3296564 (WO97/03959) was used.

TABLE 1

| | | | |
|---|---|---|---|
| Core | Crystalline cellulose (particle) | 26.0 | mg |
| First layer | Atorvastatin calcium trihydrate | 10.8 | mg |
| | SLS | 10.8 | mg |
| | HPMC | 4.3 | mg |
| Second layer | Methylcellulose | 2.6 | mg |
| Third layer | Sodium citrate | 13.7 | mg |
| | Methylcellulose | 13.7 | mg |
| Fourth layer | Methylcellulose | 4.1 | mg |

### (1) Preparation of First Layer

To a solution prepared by dissolving 208.3 g of sodium laurylsulfate (SLS) (Nikko Chemicals Co., Ltd., product name: NIKKOL SLS, the same compound was used in the following examples) and 83.4 g of hydroxypropyl methylcellulose (HPMC) (Shin-Etsu Chemical Co., Ltd., product name: TC-5E, the same compound was used in the following examples, unless otherwise specified) in 2000.0 g of purified water, 208.3 g of atorvastatin calcium trihydrate (Pfizer Inc., the same compound was used in the following examples) was added while stirring to prepare a dispersion liquid. The resulting dispersion liquid was sprayed on 500 g of crystalline cellulose (particle) (Asahi Kasei Chemicals Corporation, product name: CP-102Y, the same compound was used in the following examples) using a fluidized bed granulating apparatus (Glatt GmbH, product name: GPCG-1, the same apparatus was used in the following examples) to prepare particles coated with the first layer (Conditions for fluidized bed granulation: spray speed=7.0 g/min, air pressure of the spray=0.20 MPa).

### (2) Preparation of Second Layer

A liquid prepared by dissolving 47.5 g of methylcellulose (Shin-Etsu Chemical Co., Ltd., product name: SM-4, the same compound was used in the following examples) in 1140.0 g of purified water was sprayed on 950.0 g of the particles coated with the first layer using a fluidized bed granulating apparatus to prepare particles coated with the second layer (Conditions for fluidized bed granulation: spray speed=6.6 g/min, air pressure of the spray=0.20 MPa).

### (3) Preparation of Third Layer

A liquid prepared by dissolving 170.9 g of sodium citrate dihydrate (Wako Pure Chemical Industries, Ltd., the same compound was used in the following examples) and 150.0 g of methylcellulose in 3964.8 g of purified water was sprayed on 600.0 g of the particles coated with the second layer using a fluidized bed granulating apparatus to prepare particles coated with the third layer (Conditions for fluidized bed granulation: spray speed=6.5 g/min, air pressure of the spray=0.25 MPa).

### (4) Preparation of Fourth Layer

A liquid prepared by dissolving 45.0 g of methylcellulose in 1080.0 g of purified water was sprayed on 900.0 g of the particles coated with the third layer using a fluidized bed granulating apparatus to prepare particles coated with the fourth layer (Conditions for fluidized bed granulation: spray speed=6.6 g/min, air pressure of the spray=0.25 MPa).

TABLE 2

| | | |
|---|---|---|
| Fifth layer | Eudragit E | 7.9 mg |
| | Talc | 4.5 mg |
| | HPMC | 0.6 mg |

### (5) Preparation of Fifth Layer

An HPMC liquid (a mixed liquid of water and alcohol) was prepared by mixing 684.0 g of methanol with a solution prepared by dissolving 2.0 g of HPMC in 171.0 g of purified water. To this HPMC liquid, 27.4 g of a methyl methacrylate-butyl methacrylate-dimethylaminoethyl methacrylate copolymer (Eudragit E)(Evonik Degussa GmbH, Product name: Eudragit E100, the same compound was used in the following examples) was dissolved to prepare a solution, and then 15.7 g of talc (Matsumura Sangyo Co., Ltd., Product name: High-Filler, the same compound was used in the following examples) was dispersed to this solution. The resulting dispersion was sprayed on 300.0 g of the drug-containing particles coated with the fourth layer using a fluidized bed granulating apparatus to prepare a granular pharmaceutical composition of the present invention (Conditions for fluidized bed granulation: spray speed=7.0 g/min, air pressure of the spray=0.2 MPa).

A mixture of 557.8 g of D-mannitol (ROQUETTE, product name: PEARLITOL 50C) and 6.5 g of maltose (Hayashibara Shoji, Inc., Product name: Sunmalt S) was granulated with 258 g of a maltose solution (containing 51.6 g of maltose) using a fluidized bed granulating apparatus to prepare a granulated product for a rapidly disintegrating tablet in the buccal cavity. A mixture of 395.9 mg of the resulting granulated product and 99.0 mg of this granular pharmaceutical composition of the present invention was filled in a die having a diameter of 10.5 mm, and tabletted using an autograph (Shimadzu, AGS-20KNG, the same apparatus was used in the following examples) under various pressures (2.0 kN, 3.0 kN, and 5.0 kN) to prepare rapidly disintegrating tablets in the buccal cavity containing a granular pharmaceutical composition of the present invention.

### Example 2

TABLE 3

| | | |
|---|---|---|
| Fifth layer | Eudragit E | 15.7 mg |
| | Talc | 9.0 mg |
| | HPMC | 1.1 mg |

An HPMC liquid (a mixed liquid of water and alcohol) was prepared by mixing 1368.0 g of methanol with a solution prepared by dissolving 3.9 g of HPMC in 342.0 g of purified water. To this HPMC liquid, 54.8 g of Eudragit E was dissolved to prepare a solution, and then 31.3 g of talc was dispersed to this solution. The resulting dispersion was sprayed on 300.0 g of the drug-containing particles coated with the fourth layer prepared in Example 1 using a fluidized bed granulating apparatus to prepare a granular pharmaceutical composition of the present invention (Conditions for fluidized bed granulation: spray speed=7.0 g/min, air pressure of the spray=0.2 MPa).

A mixture of 447.5 mg of the granulated product for a rapidly disintegrating tablet in the buccal cavity prepared in Example 1 and 111.9 mg of this granular pharmaceutical composition of the present invention was filled in a die having a diameter of 10.5 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 3

TABLE 4

| | | |
|---|---|---|
| Fifth layer | Eudragit E | 21.0 mg |
| | Talc | 12.0 mg |
| | HPMC | 1.5 mg |

An HPMC liquid (a mixed liquid of water and alcohol) was prepared by mixing 1824.0 g of methanol with a solution prepared by dissolving 5.2 g of HPMC in 456.0 g of purified water. To this HPMC liquid, 73.0 g of Eudragit E was dissolved to prepare a solution, and then 41.7 g of talc was dispersed to this solution. The resulting dispersion was sprayed on 300.0 g of the drug-containing particles coated with the fourth layer prepared in Example 1 using a fluidized bed granulating apparatus to prepare a granular pharmaceutical composition of the present invention (Conditions for fluidized bed granulation: spray speed=7.0 g/min, air pressure of the spray=0.2 MPa).

A mixture of 481.9 mg of the granulated product for a rapidly disintegrating tablet in the buccal cavity prepared in Example 1 and 120.5 mg of this granular pharmaceutical composition of the present invention was filled in a die having a diameter of 10.5 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 4

TABLE 5

| | | |
|---|---|---|
| Fifth layer | Eudragit E | 7.7 mg |
| | Talc | 4.4 mg |
| | HPMC | 0.9 mg |

An HPMC liquid (a mixed liquid of water and alcohol) was prepared by mixing 684.0 g of methanol with a solution prepared by dissolving 3.1 g of HPMC in 171.0 g of purified water. To this HPMC liquid, 26.7 g of Eudragit E was dissolved to prepare a solution, and then 15.3 g of talc was dispersed to this solution. The resulting dispersion was sprayed on 300.0 g of the drug-containing particles coated with the fourth layer prepared in Example 1 using a fluidized bed granulating apparatus to prepare a granular pharmaceutical composition of the present invention (Conditions for fluidized bed granulation: spray speed=7.0 g/min, air pressure of the spray=0.18 MPa). The average particle size of the resulting masking particles was 230 μm.

A mixture of 395.9 mg of the granulated product for a rapidly disintegrating tablet in the buccal cavity prepared in Example 1 and 99.0 mg of this granular pharmaceutical composition of the present invention was filled in a die having a diameter of 11 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 5

TABLE 6

| | | |
|---|---|---|
| Fifth layer | Eudragit E | 15.3 mg |
| | Talc | 8.8 mg |
| | HPMC | 1.8 mg |

An HPMC liquid (a mixed liquid of water and alcohol) was prepared by mixing 1368.0 g of methanol with a solution prepared by dissolving 6.1 g of HPMC in 342.0 g of purified water. To this HPMC liquid, 53.4 g of Eudragit E was dissolved to prepare a solution, and then 30.5 g of talc was dispersed to this solution. The resulting dispersion was sprayed on 300.0 g of the drug-containing particles coated with the fourth layer prepared in Example 1 using a fluidized bed granulating apparatus to prepare a granular pharmaceutical composition of the present invention (Conditions for fluidized bed granulation: spray speed=7.0 g/min, air pressure of the spray=0.18 MPa). The average particle size of the resulting masking particles was 244 μm.

A mixture of 447.5 mg of the granulated product for a rapidly disintegrating tablet in the buccal cavity prepared in Example 1 and 111.9 mg of this granular pharmaceutical composition of the present invention was filled in a die having a diameter of 11 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 6

TABLE 7

| | | |
|---|---|---|
| Fifth layer | Eudragit E | 20.4 mg |
| | Talc | 11.7 mg |
| | HPMC | 2.3 mg |

An HPMC liquid (a mixed liquid of water and alcohol) was prepared by mixing 1824.0 g of methanol with a solution prepared by dissolving 8.1 g of HPMC in 456.0 g of purified water. To this HPMC liquid, 71.2 g of Eudragit E was dissolved to prepare a solution, and then 40.7 g of talc was dispersed to this solution. The resulting dispersion was sprayed on 300.0 g of the drug-containing particles coated with the fourth layer prepared in Example 1 using a fluidized bed granulating apparatus to prepare a granular pharmaceutical composition of the present invention (Conditions for fluidized bed granulation: spray speed=7.0 g/min, air pressure of the spray=0.18 MPa). The average particle size of the resulting masking particles was 251 μm.

A mixture of 481.9 mg of the granulated product for a rapidly disintegrating tablet in the buccal cavity prepared in Example 1 and 120.5 mg of this granular pharmaceutical composition of the present invention was filled in a die having a diameter of 11 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 7

TABLE 8

| | | |
|---|---|---|
| Fifth layer | Eudragit E | 7.5 mg |
| | Talc | 4.3 mg |
| | HPMC | 1.1 mg |

An HPMC liquid (a mixed liquid of water and alcohol) was prepared by mixing 684.0 g of methanol with a solution prepared by dissolving 3.8 g of HPMC in 171.0 g of purified water. To this HPMC liquid, 26.3 g of Eudragit E was dissolved to prepare a solution, and then 15.0 g of talc was dispersed to this solution. The resulting dispersion was sprayed on 300.0 g of the drug-containing particles coated with the fourth layer prepared in Example 1 using a fluidized bed granulating apparatus to prepare a granular pharmaceutical composition of the present invention (Conditions for fluidized bed granulation: spray speed=7.0 g/min, air pressure of the spray=0.18 MPa). The average particle size of the resulting masking particles was 241 μm.

A mixture of 395.9 mg of the granulated product for a rapidly disintegrating tablet in the buccal cavity prepared in Example 1 and 99.0 mg of this granular pharmaceutical composition of the present invention was filled in a die having a diameter of 10.5 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 8

TABLE 9

| | | |
|---|---|---|
| Fifth layer | Eudragit E | 15.1 mg |
| | Talc | 8.6 mg |
| | HPMC | 2.2 mg |

An HPMC liquid (a mixed liquid of water and alcohol) was prepared by mixing 1368.0 g of methanol with a solution prepared by dissolving 7.5 g of HPMC in 342.0 g of purified water. To this HPMC liquid, 52.5 g of Eudragit E was dissolved to prepare a solution, and then 30.0 g of talc was dispersed to this solution. The resulting dispersion was sprayed on 300.0 g of the drug-containing particles coated with the fourth layer prepared in Example 1 using a fluidized bed granulating apparatus to prepare a granular pharmaceutical composition of the present invention (Conditions for fluidized bed granulation: spray speed=7.0 g/min, air pressure of the spray=0.18 MPa). The average particle size of the resulting masking particles was 240 μm.

A mixture of 447.5 mg of the granulated product for a rapidly disintegrating tablet in the buccal cavity prepared in Example 1 and 111.9 mg of this granular pharmaceutical composition of the present invention was filled in a die having a diameter of 10.5 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 9

TABLE 10

| | | |
|---|---|---|
| Fifth layer | Eudragit E | 20.1 mg |
| | Talc | 11.5 mg |
| | HPMC | 2.9 mg |

An HPMC liquid (a mixed liquid of water and alcohol) was prepared by mixing 1824.0 g of methanol with a solution prepared by dissolving 10.0 g of HPMC in 456.0 g of purified water. To this HPMC liquid, 70.0 g of Eudragit E was dissolved to prepare a solution, and then 40.0 g of talc was dispersed to this solution. The resulting dispersion was sprayed on 300.0 g of the drug-containing particles coated with the fourth layer prepared in Example 1 using a fluidized bed granulating apparatus to prepare a granular pharmaceutical composition of the present invention (Conditions for fluidized bed granulation: spray speed=7.0 g/min, air pressure of the spray=0.18 MPa). The average particle size of the resulting masking particles was 248 μm.

A mixture of 481.9 mg of the granulated product for a rapidly disintegrating tablet in the buccal cavity prepared in Example 1 and 120.5 mg of this granular pharmaceutical composition of the present invention was filled in a die having a diameter of 10.5 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 10

TABLE 11

| | | |
|---|---|---|
| Core | Crystalline cellulose (particle) | 26.0 mg |
| First layer | Atorvastatin calcium trihydrate | 10.8 mg |
| | SLS | 10.8 mg |
| | HPMC | 4.3 mg |
| Second layer | Methylcellulose | 34.0 mg |
| Third layer | Eudragit E | 20.4 mg |
| | Talc | 11.7 mg |
| | HPMC | 2.3 mg |

### (1) Preparation of Second Layer

A liquid prepared by dissolving 196.1 g of methylcellulose in 4707.0 g of purified water was sprayed, using a fluidized bed granulating apparatus, on 300.0 g of the particles coated with the first layer prepared in Example 1 to prepare particles coated with the second layer (Conditions for fluidized bed granulation: spray speed=6.6 g/min, air pressure of the spray=0.25 MPa).

### (2) Preparation of Third Layer

An HPMC liquid (a mixed liquid of water and alcohol) was prepared by mixing 1824.0 g of methanol with a solution prepared by dissolving 8.1 g of HPMC in 456.0 g of purified water. To this HPMC liquid, 71.2 g of Eudragit E was dissolved to prepare a solution, and then 40.7 g of talc was dispersed to this solution. The resulting dispersion was sprayed, using a fluidized bed granulating apparatus, on 300.0 g of the drug-containing particles coated with the second layer to prepare a granular pharmaceutical composition of the present invention (Conditions for fluidized bed granulation: spray speed=7.0 g/min, air pressure of the spray=0.18 MPa). The average particle size of the resulting granular pharmaceutical composition was 247 μm.

A mixture of 481.9 mg of the granulated product for a rapidly disintegrating tablet in the buccal cavity prepared in Example 1 and 120.5 mg of this granular pharmaceutical composition of the present invention was filled in a die having a diameter of 11 mm, and tabletted using an autograph under various pressures (2.0 kN, 3.0 kN, and 5.0 kN) to prepare rapidly disintegrating tablets in the buccal cavity containing a granular pharmaceutical composition of the present invention.

### Example 11

TABLE 12

| | | |
|---|---|---|
| Core | Crystalline cellulose (particle) | 10.8 mg |
| First layer | Atorvastatin calcium trihydrate | 10.8 mg |
| | SLS | 6.5 mg |
| | HPMC | 4.3 mg |
| Second layer | Sodium citrate | 4.9 mg |
| | Methylcellulose | 4.9 mg |
| Third layer | Eudragit E | 12.1 mg |
| | Talc | 3.5 mg |
| | HPMC | 1.4 mg |

### (1) Preparation of First Layer

To a solution prepared by dissolving 180.0 g of sodium laurylsulfate and 120.0 g of HPMC in 2400.0 g of purified water, 300.0 g of atorvastatin calcium trihydrate was added while stirring to prepare a dispersion liquid. The resulting dispersion liquid was sprayed on 500.0 g of crystalline cellulose (particle) using a fluidized bed granulating apparatus to prepare particles coated with the first layer (Conditions for fluidized bed granulation: spray speed=6.0 g/min, air pressure of the spray=0.20 MPa).

### (2) Preparation of Second Layer

A liquid prepared by dissolving 114.5 g of sodium citrate dihydrate and 100.5 g of methylcellulose in 2656.4 g of purified water was sprayed, using a fluidized bed granulating apparatus, on 670.0 g of the particles coated with the first layer to prepare particles coated with the second layer (Conditions for fluidized bed granulation: spray speed=7.0 g/min, air pressure of the spray=0.28 MPa).

### (3) Preparation of Third Layer

An HPMC liquid (a mixed liquid of water and alcohol) was prepared by mixing 1727.4 g of methanol with a solution prepared by dissolving 9.28 g of HPMC in 432.0 g of purified water. To this HPMC liquid, 81.2 g of Eudragit E was dissolved to prepare a solution, and then 23.2 g of talc was dispersed to this solution. The resulting dispersion was sprayed, using a fluidized bed granulating apparatus, on 284.2 g of the particles coated with the second layer to prepare a granular pharmaceutical composition of the present invention (Conditions for fluidized bed granulation: spray speed=7.0 g/min, air pressure of the spray=0.20 MPa).

### (4) Preparation of Fourth Layer

A mannitol solution, which had been prepared by dissolving 15.0 g of mannitol in 135.0 g of purified water, was sprayed using a fluidized bed granulating apparatus on 300.0 g of the particles coated with the third layer to prepare a granular pharmaceutical composition (masking particles) of the present invention (Conditions for fluidized bed granulation: spray speed=5.0 g/min, air pressure of the spray=0.18 MPa).

A mixture of 248.8 mg of the granulated product for a rapidly disintegrating tablet in the buccal cavity prepared in Example 1, 3.1 mg of magnesium stearate (Merck), and 62.2 mg of the resulting masking particles was filled in a die having a diameter of 9.5 mm, and tabletted using an autograph under a pressure of 2.0 kN to prepare a rapidly disintegrating tablet in the buccal cavity.

### Example 12

TABLE 13

| | | |
|---|---|---|
| Core | Crystalline cellulose (particle) | 10.8 mg |
| First layer | Atorvastatin calcium trihydrate | 10.8 mg |
| | SLS | 6.5 mg |
| | HPMC | 4.3 mg |
| Second layer | Sodium citrate | 4.9 mg |
| | Methylcellulose | 4.9 mg |
| Third layer | Eudragit E | 12.3 mg |
| | Talc | 3.5 mg |
| | HPMC | 1.1 mg |

### (1) Preparation of Third Layer

An HPMC liquid (a mixed liquid of water and alcohol) was prepared by mixing 1760.2 g of methanol with a solution prepared by dissolving 7.8 g of HPMC in 440.0 g of purified water. To this HPMC liquid, 84.0 g of Eudragit E was dissolved to prepare a solution, and then 24.0 g of talc was dispersed to this solution. The resulting dispersion was sprayed, using a fluidized bed granulating apparatus, on 289.5 g of the particles coated with the second layer prepared in Example 11 to prepare a granular pharmaceutical composition of the present invention (Conditions for fluidized bed granulation: spray speed=7.0 g/min, air pressure of the spray=0.20 MPa).

### (4) Preparation of Fourth Layer

A mannitol solution, which had been prepared by dissolving 15.0 g of mannitol in 135.0 g of purified water, was sprayed using a fluidized bed granulating apparatus on 300.0 g of the particles coated with the third layer to prepare a granular pharmaceutical composition (masking particles) of the present invention (Conditions for fluidized bed granulation: spray speed=5.0 g/min, air pressure of the spray=0.18 MPa).

A mixture of 248.4 mg of the granulated product for a rapidly disintegrating tablet in the buccal cavity prepared in Example 1, 3.2 mg of magnesium stearate, and 62.1 mg of the resulting masking particles was filled in a die having a diameter of 9.5 mm, and tabletted using an autograph under a pressure of 2.0 kN to prepare a rapidly disintegrating tablet in the buccal cavity.

### Example 13

TABLE 14

| | | |
|---|---|---|
| Core | Crystalline cellulose (particle) | 10.8 mg |
| First layer | Atorvastatin calcium trihydrate | 10.8 mg |
| | SLS | 6.5 mg |
| | HPMC | 4.3 mg |
| Second layer | Sodium citrate | 4.9 mg |
| | Methylcellulose | 4.9 mg |
| Third layer | Eudragit E | 10.0 mg |
| | Talc | 5.7 mg |
| | HPMC | 1.2 mg |

### (1) Preparation of First Layer

To a solution prepared by dissolving 3.25 kg of sodium laurylsulfate and 2.17 kg of HPMC in 43.36 kg of purified water, 5.42 kg of atorvastatin calcium trihydrate was added while stirring to prepare a dispersion liquid. The resulting dispersion liquid was sprayed on 5.42 kg of crystalline cellulose (particle) using a fluidized bed granulating apparatus to prepare particles coated with the first layer (Conditions for fluidized bed granulation: spray speed=80 g/min, air pressure of the spray=0.35 MPa).

### (2) Preparation of Second Layer

A liquid prepared by dissolving 1.33 kg of sodium citrate dihydrate and 1.17 kg of methylcellulose in 30.96 kg of purified water was sprayed, using a fluidized bed granulating apparatus, on 7.8 kg of the particles coated with the first layer to prepare particles coated with the second layer (Conditions for fluidized bed granulation: spray speed=90 g/min, air pressure of the spray=0.35 MPa).

### (3) Preparation of Third Layer

To a mixed liquid consisting of 15.67 kg of purified water and 62.67 kg of methanol, 0.28 kg of HPMC, 2.45 kg of Eudragit E, and 1.4 kg of talc were added to prepare a dispersion. The resulting dispersion was sprayed, using a fluidized bed granulating apparatus, on 10.30 kg of the particles coated with the second layer to prepare a granular pharmaceutical composition of the present invention (Conditions for fluidized bed granulation: spray speed=90 g/min, air pressure of the spray=0.45 MPa).

### (4) Preparation of Fourth Layer

A mannitol solution, which had been prepared by dissolving 0.72 kg of mannitol in 6.28 kg of purified water, was sprayed using a fluidized bed granulating apparatus on 14.43 kg of the particles coated with the third layer to prepare a granular pharmaceutical composition (masking particles) of the present invention (Conditions for fluidized bed granulation: spray speed=100 g/min, air pressure of the spray=0.35 MPa).

A mixture of 236.9 mg of the granulated product for a rapidly disintegrating tablet in the buccal cavity prepared in Example 1 and 63.1 mg of the resulting masking particles was filled in a die having a diameter of 9.5 mm, and tabletted using an autograph under various pressures (2.0 kN and 3.0 kN) to prepare rapidly disintegrating tablets in the buccal cavity.

### Example 14

TABLE 15

| | | |
|---|---|---|
| Core | Crystalline cellulose (particle) | 10.8 mg |
| First layer | Atorvastatin calcium trihydrate | 10.8 mg |
| | SLS | 6.5 mg |
| | HPMC | 4.3 mg |
| Second layer | Sodium citrate | 4.9 mg |
| | Methylcellulose | 4.9 mg |
| Third layer | Eudragit E | 10.0 mg |
| | Talc | 5.7 mg |
| | HPMC(TC-5S) | 1.2 mg |

### (1) Preparation of Second Layer

A liquid prepared by dissolving 121.4 g of sodium citrate dihydrate and 106.5 g of methylcellulose in 2815 g of purified water was sprayed, using a fluidized bed granulating apparatus, on 710.0 g of the particles coated with the first layer prepared in Example 13 to prepare particles coated with the second layer (Conditions for fluidized bed granulation: spray speed=6.0 g/min, air pressure of the spray=0.20 MPa).

### (2) Preparation of Third Layer

An HPMC liquid (a mixed liquid of water and alcohol) was prepared by mixing 1824.0 g of methanol with a solution prepared by dissolving 8.1 g of HPMC (Shin-Etsu Chemical Co., Ltd., product name: TC-5S) in 456.0 g of purified water. To this HPMC liquid, 71.2 g of Eudragit E was dissolved to prepare a solution, and then 40.7 g of talc was dispersed to this solution. The resulting dispersion was sprayed, using a fluidized bed granulating apparatus, on 300.0 g of the particles coated with the second layer to prepare a granular pharmaceutical composition of the present invention (Conditions for fluidized bed granulation: spray speed=6.2 g/min, air pressure of the spray=0.22 MPa).

A mixture of 225.0 mg of the granulated product for a rapidly disintegrating tablet in the buccal cavity prepared in Example 1 and 59.2 mg of the resulting masking particles was filled in a die having a diameter of 9.5 mm, and tabletted using an autograph under various pressures (2.0 kN and 3.0 kN) to prepare rapidly disintegrating tablets in the buccal cavity.

### Example 15

TABLE 16

| | | |
|---|---|---|
| Core | Crystalline cellulose (particle) | 10.8 mg |
| First layer | Atorvastatin calcium trihydrate | 10.8 mg |
| | SLS | 6.5 mg |
| | HPMC | 4.3 mg |
| Second layer | Sodium citrate | 4.9 mg |
| | Methylcellulose | 4.9 mg |
| Third layer | Eudragit E | 10.0 mg |
| | Talc | 5.7 mg |
| | HPC-SL | 1.2 mg |

### (1) Preparation of Third Layer

An HPMC liquid (a mixed liquid of water and alcohol) was prepared by mixing 1824.0 g of methanol with a solution prepared by dissolving 8.1 g of hydroxypropyl cellulose (Nippon Soda Co., Ltd., product name: HPC-SL) in 456.0 g of purified water. To this HPMC liquid, 71.2 g of Eudragit E was dissolved to prepare a solution, and then 40.7 g of talc was dispersed to this solution. The resulting dispersion was sprayed, using a fluidized bed granulating apparatus, on 300.0 g of the particles coated with the second layer prepared in Example 14 to prepare a granular pharmaceutical composition of the present invention (Conditions for fluidized bed granulation: spray speed=7.0 g/min, air pressure of the spray=0.18 MPa).

A mixture of 225.0 mg of the granulated product for a rapidly disintegrating tablet in the buccal cavity prepared in Example 1 and 59.2 mg of the resulting masking particles was filled in a die having a diameter of 9.5 mm, and tabletted using an autograph under various pressures (2.0 kN and 3.0 kN) to prepare rapidly disintegrating tablets in the buccal cavity.

### Example 16

TABLE 17

| | | |
|---|---|---|
| Core | Crystalline cellulose (particle) | 10.8 mg |
| First layer | Atorvastatin calcium trihydrate | 10.8 mg |
| | SLS | 6.5 mg |
| | HPMC | 4.3 mg |
| Second layer | Sodium citrate | 4.9 mg |
| | Methylcellulose | 4.9 mg |
| Third layer | Eudragit E | 10.0 mg |
| | Talc | 5.7 mg |
| | PVA(EG-05) | 1.2 mg |

### (1) Preparation of Third Layer

An HPMC liquid (a mixed liquid of water and alcohol) was prepared by mixing 1824.0 g of methanol with a solution prepared by dissolving 8.1 g of polyvinyl alcohol (The Nippon Synthetic Chemical Industry Co., Ltd., product name: GOHSENOL EG-05) in 456.0 g of purified water. To this HPMC liquid, 71.2 g of Eudragit E was dissolved to prepare a solution, and then 40.7 g of talc was dispersed to this solution. The resulting dispersion was sprayed, using a fluidized bed granulating apparatus, on 300.0 g of the particles coated with the second layer prepared in Example 14 to prepare a granular pharmaceutical composition of the present invention (Conditions for fluidized bed granulation: spray speed=7.0 g/min, air pressure of the spray=0.22 MPa).

A mixture of 225.0 mg of the granulated product for a rapidly disintegrating tablet in the buccal cavity prepared in Example 1 and 59.2 mg of the resulting masking particles was filled in a die having a diameter of 9.5 mm, and tabletted using an autograph under various pressures (2.0 kN and 3.0 kN) to prepare rapidly disintegrating tablets in the buccal cavity.

### Example 17

TABLE 18

| | | |
|---|---|---|
| Core | Crystalline cellulose (particle) | 10.8 mg |
| First layer | Atorvastatin calcium trihydrate | 10.8 mg |
| | SLS | 6.5 mg |
| | HPMC | 4.3 mg |
| Second layer | Sodium citrate | 4.9 mg |
| | Methylcellulose | 4.9 mg |
| Third layer | Eudragit E | 10.0 mg |
| | Talc | 5.7 mg |
| | Kollicoat IR | 1.2 mg |

### (1) Preparation of Third Layer

An HPMC liquid (a mixed liquid of water and alcohol) was prepared by mixing 1824.0 g of methanol with a solution prepared by dissolving 8.1 g of a polyvinyl alcohol-polyethylene glycol graft copolymer (BASF, product name: Kollicoat IR) in 456.0 g of purified water. To this HPMC liquid, 71.2 g of Eudragit E was dissolved to prepare a solution, and then 40.7 g of talc was dispersed to this solution. The resulting dispersion was sprayed, using a fluidized bed granulating apparatus, on 300.0 g of the particles coated with the second layer prepared in Example 14 to prepare a granular pharmaceutical composition of the present invention (Conditions for fluidized bed granulation: spray speed=7.0 g/min, air pressure of the spray=0.18 MPa).

A mixture of 225.0 mg of the granulated product for a rapidly disintegrating tablet in the buccal cavity prepared in Example 1 and 59.2 mg of the resulting masking particles was filled in a die having a diameter of 9.5 mm, and tabletted using an autograph under various pressures (2.0 kN and 3.0 kN) to prepare rapidly disintegrating tablets in the buccal cavity.

### Example 18

TABLE 19

| | | |
|---|---|---|
| Core | Crystalline cellulose (particle) | 10.8 mg |
| First layer | Atorvastatin calcium trihydrate | 10.8 mg |
| | SLS | 6.5 mg |
| | HPMC | 4.3 mg |
| Second layer | Sodium citrate | 4.9 mg |
| | Methylcellulose | 4.9 mg |
| Third layer | Eudragit E | 10.0 mg |
| | Talc | 5.7 mg |
| | PVP-K30 | 1.2 mg |

### (1) Preparation of Third Layer

An HPMC liquid (a mixed liquid of water and alcohol) was prepared by mixing 1824.0 g of methanol with a solution prepared by dissolving 8.1 g of polyvinylpyrrolidone (Wako Pure Chemical Industries, Ltd., product name: Polyvinylpyrrolidone K30) in 456.0 g of purified water. To this HPMC liquid, 71.2 g of Eudragit E was dissolved to prepare a solution, and then 40.7 g of talc was dispersed to this solution. The resulting dispersion was sprayed, using a fluidized bed granulating apparatus, on 300.0 g of the particles coated with the second layer prepared in Example 14 to prepare a granular pharmaceutical composition of the present invention (Conditions for fluidized bed granulation: spray speed=7.0 g/min, air pressure of the spray=0.18 MPa).

A mixture of 225.0 mg of the granulated product for a rapidly disintegrating tablet in the buccal cavity prepared in Example 1 and 59.2 mg of the resulting masking particles was filled in a die having a diameter of 9.5 mm, and tabletted using an autograph under various pressures (2.0 kN and 3.0 kN) to prepare rapidly disintegrating tablets in the buccal cavity.

### Example 19

TABLE 20

| | | |
|---|---|---|
| Core | Crystalline cellulose (particle) | 10.0 mg |
| First layer | Flurbiprofen | 10.0 mg |
| | SLS | 6.0 mg |
| | HPMC | 4.0 mg |
| Second layer | Sodium citrate | 4.5 mg |
| | Methylcellulose | 4.5 mg |
| Third layer | Eudragit E | 9.3 mg |
| | Talc | 5.3 mg |
| | HPMC | 1.1 mg |

### (1) Preparation of First Layer

To a solution prepared by dissolving 180.0 g of sodium laurylsulfate and 120.0 g of HPMC in 2400.0 g of purified water, 300.0 g of flurbiprofen is added while stirring to prepare a dispersion liquid. The resulting dispersion liquid is sprayed on 300.0 g of crystalline cellulose (particle) using a fluidized bed granulating apparatus to prepare particles coated with the first layer (Conditions for fluidized bed granulation: spray speed=6.0 g/min, air pressure of the spray=0.20 MPa).

### (2) Preparation of Second Layer

A liquid prepared by dissolving 114.5 g of sodium citrate dihydrate and 100.5 g of methylcellulose in 2656.4 g of purified water is sprayed, using a fluidized bed granulating apparatus, on 670.0 g of the particles coated with the first layer to prepare particles coated with the second layer (Conditions for fluidized bed granulation: spray speed=7.0 g/min, air pressure of the spray=0.28 MPa).

### (3) Preparation of Third Layer

An HPMC liquid (a mixed liquid of water and alcohol) is prepared by mixing 1824.0 g of methanol with a solution prepared by dissolving 8.1 g of HPMC in 456.0 g of purified water. To this HPMC liquid, 71.2 g of Eudragit E is dissolved to prepare a solution, and then 40.7 g of talc is dispersed to this solution. The resulting dispersion is sprayed, using a fluidized bed granulating apparatus, on 300.0 g of the particles coated with the second layer to prepare a granular pharmaceutical composition of the present invention (Conditions for fluidized bed granulation: spray speed=7.0 g/min, air pressure of the spray=0.20 MPa).

A mixture of 218.4 mg of the granulated product for a rapidly disintegrating tablet in the buccal cavity prepared in Example 1 and 54.6 mg of the resulting masking particles is filled in a die having a diameter of 9.0 mm, and tabletted using an autograph under a pressure of 2.0 kN to prepare rapidly disintegrating tablets in the buccal cavity.

### Example 20

TABLE 21

| | | |
|---|---|---|
| Core | Crystalline cellulose (particle) | 10.0 mg |
| First layer | Ibuprofen | 10.0 mg |
| | SLS | 6.0 mg |
| | HPMC | 4.0 mg |
| Second layer | Sodium citrate | 4.5 mg |
| | Methylcellulose | 4.5 mg |
| Third layer | Eudragit E | 9.3 mg |
| | Talc | 5.3 mg |
| | HPMC | 1.1 mg |

### (1) Preparation of First Layer

To a solution prepared by dissolving 180.0 g of sodium laurylsulfate and 120.0 g of HPMC in 2400.0 g of purified water, 300.0 g of ibuprofen is added while stirring to prepare a dispersion liquid. The resulting dispersion liquid is sprayed on 300.0 g of crystalline cellulose (particle) using a fluidized bed granulating apparatus to prepare particles coated with the first layer (Conditions for fluidized bed granulation: spray speed=6.0 g/min, air pressure of the spray=0.20 MPa).

### (2) Preparation of Second Layer

A liquid prepared by dissolving 114.5 g of sodium citrate dihydrate and 100.5 g of methylcellulose in 2656.4 g of purified water is sprayed, using a fluidized bed granulating apparatus, on 670.0 g of the particles coated with the first layer to prepare particles coated with the second layer (Conditions for fluidized bed granulation: spray speed=7.0 g/min, air pressure of the spray=0.28 MPa).

### (3) Preparation of Third Layer

An HPMC liquid (a mixed liquid of water and alcohol) is prepared by mixing 1824.0 g of methanol with a solution prepared by dissolving 8.1 g of HPMC in 456.0 g of purified water. To this HPMC liquid, 71.2 g of Eudragit E is dissolved to prepare a solution, and then 40.7 g of talc is dispersed to this solution. The resulting dispersion is sprayed, using a fluidized bed granulating apparatus, on 300.0 g of the particles coated with the second layer to prepare a granular pharmaceutical composition of the present invention (Conditions for fluidized bed granulation: spray speed=7.0 g/min, air pressure of the spray=0.20 MPa).

A mixture of 218.4 mg of the granulated product for a rapidly disintegrating tablet in the buccal cavity prepared in Example 1 and 54.6 mg of the resulting masking particles is filled in a die having a diameter of 9.0 mm, and tabletted using an autograph under a pressure of 2.0 kN to prepare rapidly disintegrating tablets in the buccal cavity.

### Example 21

TABLE 22

| | | |
|---|---|---|
| Core | Crystalline cellulose (particle) | 10.0 mg |
| First layer | Naproxen | 10.0 mg |
| | SLS | 6.0 mg |
| | HPMC | 4.0 mg |
| Second layer | Sodium citrate | 4.5 mg |
| | Methylcellulose | 4.5 mg |
| Third layer | Eudragit E | 9.3 mg |
| | Talc | 5.3 mg |
| | HPMC | 1.1 mg |

### (1) Preparation of First Layer

To a solution prepared by dissolving 180.0 g of sodium laurylsulfate and 120.0 g of HPMC in 2400.0 g of purified water, 300.0 g of naproxen is added while stirring to prepare a dispersion liquid. The resulting dispersion liquid is sprayed on 300.0 g of crystalline cellulose (particle) using a fluidized bed granulating apparatus to prepare particles coated with the first layer (Conditions for fluidized bed granulation: spray speed=6.0 g/min, air pressure of the spray=0.20 MPa).

### (2) Preparation of Second Layer

A liquid prepared by dissolving 114.5 g of sodium citrate dihydrate and 100.5 g of methylcellulose in 2656.4 g of purified water is sprayed, using a fluidized bed granulating apparatus, on 670.0 g of the particles coated with the first layer to prepare particles coated with the second layer (Conditions for fluidized bed granulation: spray speed=7.0 g/min, air pressure of the spray=0.28 MPa).

### (3) Preparation of Third Layer

An HPMC liquid (a mixed liquid of water and alcohol) is prepared by mixing 1824.0 g of methanol with a solution prepared by dissolving 8.1 g of HPMC in 456.0 g of purified water. To this HPMC liquid, 71.2 g of Eudragit E is dissolved to prepare a solution, and then 40.7 g of talc is dispersed to this solution. The resulting dispersion is sprayed, using a fluidized bed granulating apparatus, on 300.0 g of the particles coated with the second layer to prepare a granular pharmaceutical composition of the present invention (Conditions for fluidized bed granulation: spray speed=7.0 g/min, air pressure of the spray=0.20 MPa).

A mixture of 218.4 mg of the granulated product for a rapidly disintegrating tablet in the buccal cavity prepared in Example 1 and 54.6 mg of the resulting masking particles is filled in a die having a diameter of 9.0 mm, and tabletted using an autograph under a pressure of 2.0 kN to prepare rapidly disintegrating tablets in the buccal cavity.

### Example 22

TABLE 23

| | | |
|---|---|---|
| Core | Crystalline cellulose (particle) | 10.0 mg |
| First layer | Ketoprofen | 10.0 mg |
| | SLS | 6.0 mg |
| | HPMC | 4.0 mg |
| Second layer | Sodium citrate | 4.5 mg |
| | Methylcellulose | 4.5 mg |
| Third layer | Eudragit E | 9.3 mg |
| | Talc | 5.3 mg |
| | HPMC | 1.1 mg |

### (1) Preparation of First Layer

To a solution prepared by dissolving 180.0 g of sodium laurylsulfate and 120.0 g of HPMC in 2400.0 g of purified water, 300.0 g of ketoprofen is added while stirring to prepare a dispersion liquid. The resulting dispersion liquid is sprayed on 300.0 g of crystalline cellulose (particle) using a fluidized bed granulating apparatus to prepare particles coated with the first layer (Conditions for fluidized bed granulation: spray speed=6.0 g/min, air pressure of the spray=0.20 MPa).

### (2) Preparation of Second Layer

A liquid prepared by dissolving 114.5 g of sodium citrate dihydrate and 100.5 g of methylcellulose in 2656.4 g of purified water is sprayed, using a fluidized bed granulating apparatus, on 670.0 g of the particles coated with the first layer to prepare particles coated with the second layer (Conditions for fluidized bed granulation: spray speed=7.0 g/min, air pressure of the spray=0.28 MPa).

### (3) Preparation of Third Layer

An HPMC liquid (a mixed liquid of water and alcohol) is prepared by mixing 1824.0 g of methanol with a solution prepared by dissolving 8.1 g of HPMC in 456.0 g of purified water. To this HPMC liquid, 71.2 g of Eudragit E is dissolved to prepare a solution, and then 40.7 g of talc is dispersed to this solution. The resulting dispersion is sprayed, using a fluidized bed granulating apparatus, on 300.0 g of the particles coated with the second layer to prepare a granular pharmaceutical composition of the present invention (Conditions for fluidized bed granulation: spray speed=7.0 g/min, air pressure of the spray=0.20 MPa).

A mixture of 218.4 mg of the granulated product for a rapidly disintegrating tablet in the buccal cavity prepared in Example 1 and 54.6 mg of the resulting masking particles is filled in a die having a diameter of 9.0 mm, and tabletted using an autograph under a pressure of 2.0 kN to prepare rapidly disintegrating tablets in the buccal cavity.

### Comparative Example 1

TABLE 24

| | | |
|---|---|---|
| Fifth layer | Eudragit E | 8.6 mg |
| | Talc | 4.3 mg |

To a mixed liquid consisting of 171.0 g of purified water and 684.0 g of methanol, 30.0 g of Eudragit E was dissolved, and then, 15.0 g of talc was added to prepare a dispersion. The resulting dispersion was sprayed, using a fluidized bed granulating apparatus, on 300.0 g of the drug-containing particles coated with the fourth layer prepared in Example 1 to prepare a granular pharmaceutical composition of Comparative Example 1 (Conditions for fluidized bed granulation: spray speed=7.0 g/min, air pressure of the spray=0.18 MPa).

A mixture of 395.9 mg of the granulated product for a rapidly disintegrating tablet in the buccal cavity prepared in Example 1 and 99.0 mg of the granular pharmaceutical composition of Comparative Example 1 was filled in a die having a diameter of 11 mm, and tabletted using an autograph under various pressures (2.0 kN, 3.0 kN, and 5.0 kN) to prepare rapidly disintegrating tablets in the buccal cavity containing the granular pharmaceutical composition of Comparative Example 1.

### Comparative Example 2

TABLE 25

| | | |
|---|---|---|
| Fifth layer | Eudragit E | 7.7 mg |
| | Talc | 4.4 mg |
| | Triacetin | 0.8 mg |

A triacetin liquid (a mixed liquid of water and alcohol) was prepared by adding 2.7 g of triacetin to a mixed liquid of 171.0 g of purified water and 684.0 g of methanol. To this triacetin liquid, 26.9 g of Eudragit E was dissolved, and then, 15.4 g of talc was added to prepare a dispersion. The resulting dispersion was sprayed, using a fluidized bed granulating apparatus, on 300.0 g of the drug-containing particles coated with the fourth layer prepared in Example 1 to prepare a granular pharmaceutical composition of Comparative Example 2 (Conditions for fluidized bed granulation: spray speed=8.0 g/min, air pressure of the spray=0.20 MPa). The average particle size of the resulting masking particles was 219 μm.

A mixture of 395.9 mg of the granulated product for a rapidly disintegrating tablet in the buccal cavity prepared in Example 1 and 99.0 mg of the granular pharmaceutical composition of Comparative Example 2 was filled in a die having a diameter of 10.5 mm, and tabletted using an autograph under various pressures (2.0 kN, 3.0 kN, and 5.0 kN) to prepare rapidly disintegrating tablets in the buccal cavity containing the granular pharmaceutical composition of Comparative Example 2.

### Comparative Example 3

TABLE 26

| | | |
|---|---|---|
| Core | Crystalline cellulose (particle) | 16.7 mg |
| First layer | Solifenacin succinate | 10.0 mg |
| | Macrogol 6000 | 3.3 mg |
| Second layer | NaH₂PO₄•2H₂O | 7.5 mg |
| | Methylcellulose | 7.5 mg |
| Third layer | Eudragit NE | 4.5 mg |
| | Talc | 2.6 mg |

### (1) First Layer

In a mixed liquid consisting of 552.6 g of methanol and 552.6 g of purified water, 333.3 g of solifenacin succinate and 111.1 g of Macrogol 6000 (Sanyo Chemical Industries, Ltd., the same compound was used in the following examples) were dissolved to prepare a drug solution. The resulting drug solution was sprayed on 555.6 g of crystalline cellulose (particle) using a fluidized bed granulating apparatus to prepare particles coated with the first layer (Conditions for fluidized bed granulation: spray speed=6.1 g/min, air pressure of the spray=0.24 MPa).

### (2) Second Layer

A solution prepared by dissolving 216.7 g of sodium dihydrogen phosphate dihydrate (KANTO CHEMICAL CO., INC.) and 166.7 g of methylcellulose in 4378.1 of purified water was sprayed, using a fluidized bed granulating apparatus, on 666.6 g of the particles coated with the first layer to prepare particles coated with the first layer (Conditions for fluidized bed granulation: spray speed=7.2 g/min, air pressure of the spray=0.20 MPa).

### (3) Third Layer

To a mixed liquid consisting of 33.3 g of Eudragit NE 30D and 356.7 g of purified water, 10.0 g of talc was dispersed. The resulting dispersion was sprayed, using a fluidized bed granulating apparatus, on 200.0 g of the particles coated with the second layer to prepare a granular pharmaceutical composition of Comparative Example 3 (Conditions for fluidized bed granulation: spray speed=7.0 g/min, air pressure of the spray=0.20 MPa).

A mixture of 255.0 mg of the granulated product for a rapidly disintegrating tablet in the buccal cavity prepared in Example 1 and 45.0 mg of the granular pharmaceutical composition of Comparative Example 3 was filled in a die having a diameter of 9.5 mm, and tabletted using an autograph under various pressures (2.0 kN and 3.0 kN) to prepare rapidly disintegrating tablets in the buccal cavity containing the granular pharmaceutical composition of Comparative Example 3.

### Comparative Example 4

TABLE 27

| | | |
|---|---|---|
| Core | Crystalline cellulose (particle) | 21.7 mg |
| First layer | Atorvastatin calcium trihydrate | 10.8 mg |
| | SLS | 6.5 mg |
| | HPMC | 4.3 mg |
| Second layer | Methylcellulose | 2.2 mg |
| Third layer | Sodium citrate | 11.4 mg |
| | Methylcellulose | 11.4 mg |
| Fourth layer | Methylcellulose | 3.4 mg |
| Fifth layer | Eudragit E | 11.0 mg |
| | Talc | 6.3 mg |
| Sixth layer | HPMC | 8.9 mg |

### (1) Preparation of First Layer

To a solution prepared by dissolving 135.0 g of sodium laurylsulfate and 90.0 g of HPMC in 1800.0 g of purified water, 225.0 g of atorvastatin calcium trihydrate was added while stirring to prepare a dispersion liquid. The resulting dispersion liquid was sprayed on 500 g of crystalline cellulose (particle) using a fluidized bed granulating apparatus to prepare particles coated with the first layer (Conditions for fluidized bed granulation: spray speed=7.0 g/min, air pressure of the spray=0.20 MPa).

### (2) Preparation of Second Layer

A liquid prepared by dissolving 40.0 g of methylcellulose in 960.0 g of purified water was sprayed, using a fluidized bed granulating apparatus, on 800.0 g of the particles coated with the first layer to prepare particles coated with the second layer (Conditions for fluidized bed granulation: spray speed=5.0 g/min, air pressure of the spray=0.23 MPa).

### (3) Preparation of Third Layer

A liquid prepared by dissolving 114.0 g of sodium citrate dihydrate and 100.0 g of methylcellulose in 2643.2 g of purified water was sprayed, using a fluidized bed granulating apparatus, on 400 g of the particles coated with the second layer to prepare particles coated with the third layer (Conditions for fluidized bed granulation: spray speed=7.0 g/min, air pressure of the spray=0.25 MPa).

### (4) Preparation of Fourth Layer

A liquid prepared by dissolving 30.0 g of methylcellulose in 720.0 g of purified water was sprayed, using a fluidized bed granulating apparatus, on 600.0 g of the particles coated with the third layer to prepare particles coated with the fourth layer (Conditions for fluidized bed granulation: spray speed=6.6 g/min, air pressure of the spray=0.25 MPa).

### (5) Preparation of Fifth Layer

In a mixed liquid consisting of 136.8 g of purified water and 1231.2 g of methanol, 45.8 g of Eudragit E was dissolved, and then, 26.2 g of talc was dispersed. The resulting dispersion was sprayed, using a fluidized bed granulating apparatus, on 300.0 g of the particles coated with the fourth layer (Conditions for fluidized bed granulation: spray speed=5.2 g/min, air pressure of the spray=0.22 MPa).

### (6) Preparation of Sixth Layer

An HPMC solution, which had been prepared by dissolving 25.0 g of HPMC in 600.0 g of purified water, was sprayed using a fluidized bed granulating apparatus on 250.0 g of the drug-containing particles coated with the fifth layer to prepare a granular pharmaceutical composition of Comparative Example 4 (Conditions for fluidized bed granulation: spray speed=5.0 g/min, air pressure of the spray=0.20 MPa).

A mixture of 391.2 mg of the granulated product for a rapidly disintegrating tablet in the buccal cavity prepared in Example 1 and 99.0 mg of the granular pharmaceutical composition of Comparative Example 4 was filled in a die having a diameter of 10.5 mm, and tabletted using an autograph under various pressures (2.0 kN, 3.0 kN, and 5.0 kN) to prepare rapidly disintegrating tablets in the buccal cavity containing the granular pharmaceutical composition of Comparative Example 4.

### Experimental Example 1

### [Dissolution Test of Granular Pharmaceutical Compositions]

With respect to the granular pharmaceutical compositions (before tabletting) prepared in Examples 4-14 and 16-18 and Comparative Examples 1-3 and the rapidly disintegrating tablets in the buccal cavity containing the same (after tabletting), a dissolution test was carried out using a 6-unit automatic dissolution test apparatus in accordance with the dissolution test, method 2 described in the Japanese Pharmacopoeia. As a control sample, particles containing 10 mg of the drug was weighed out. As a test fluid, 900 mL of water was used, except that 900 mL of the second fluid (JP2) described in this dissolution test of the Japanese Pharmacopoeia was used for the test samples prepared in Comparative Example 3. The paddle rotation speed was 100 rpm.

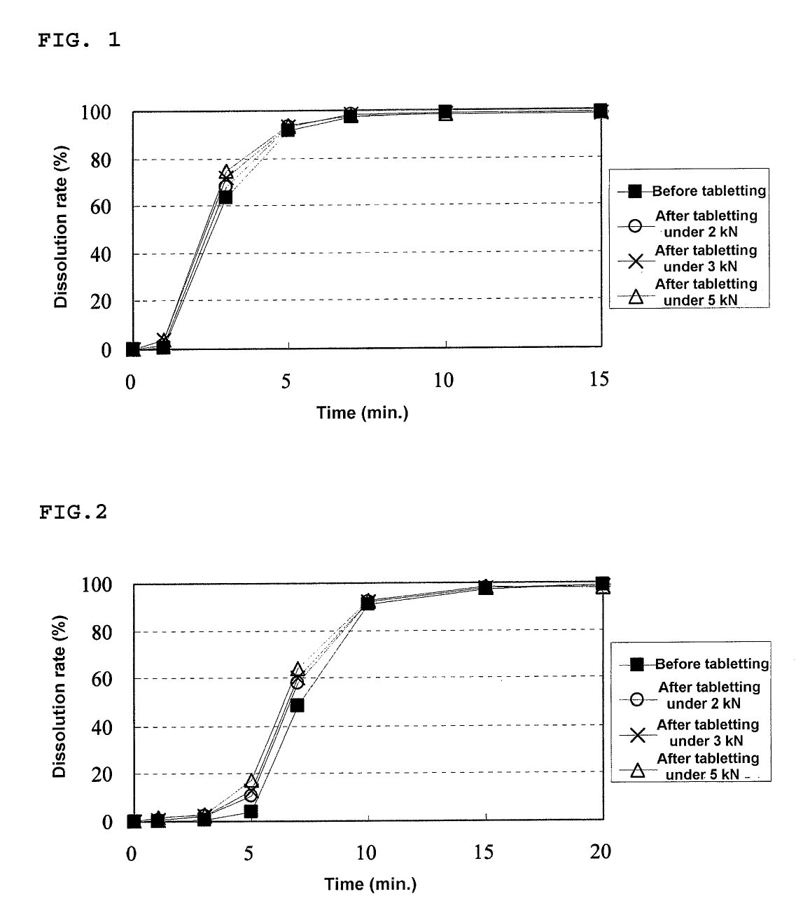

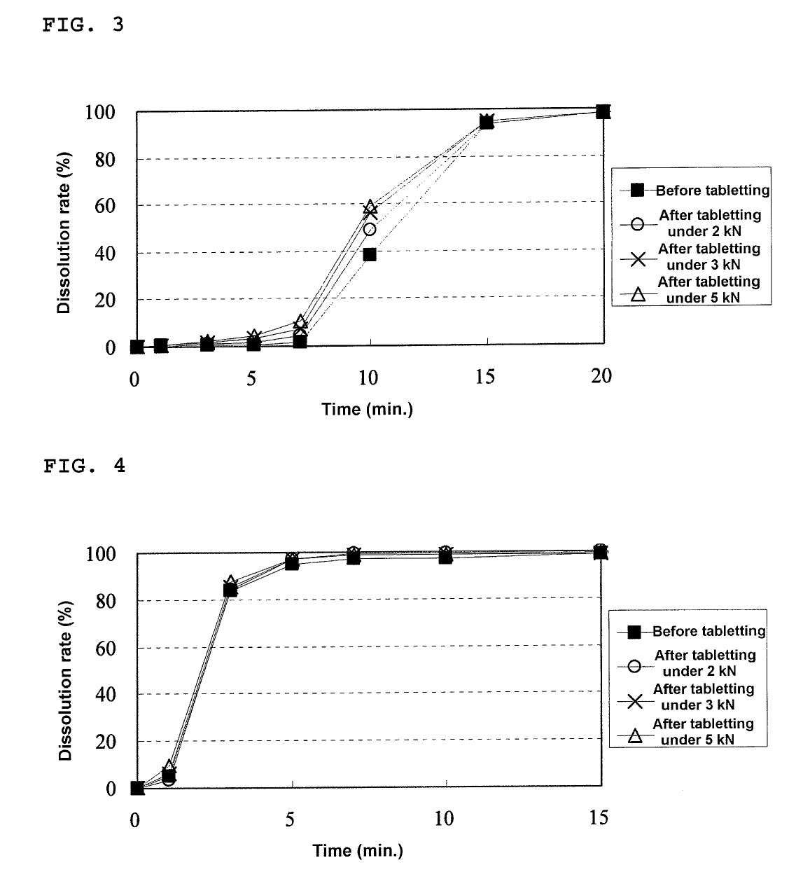

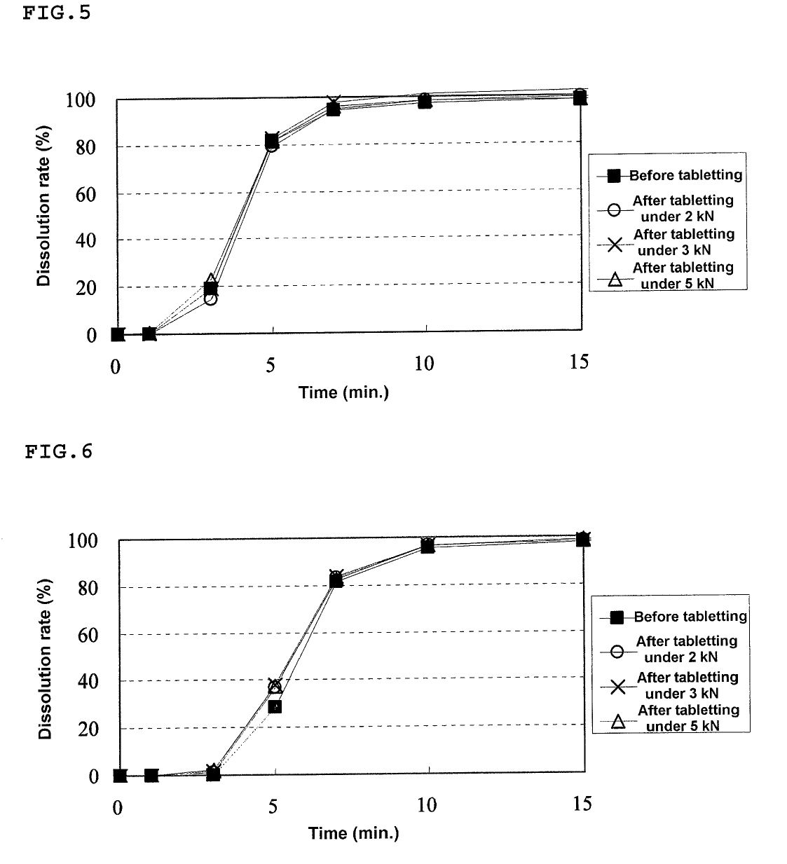

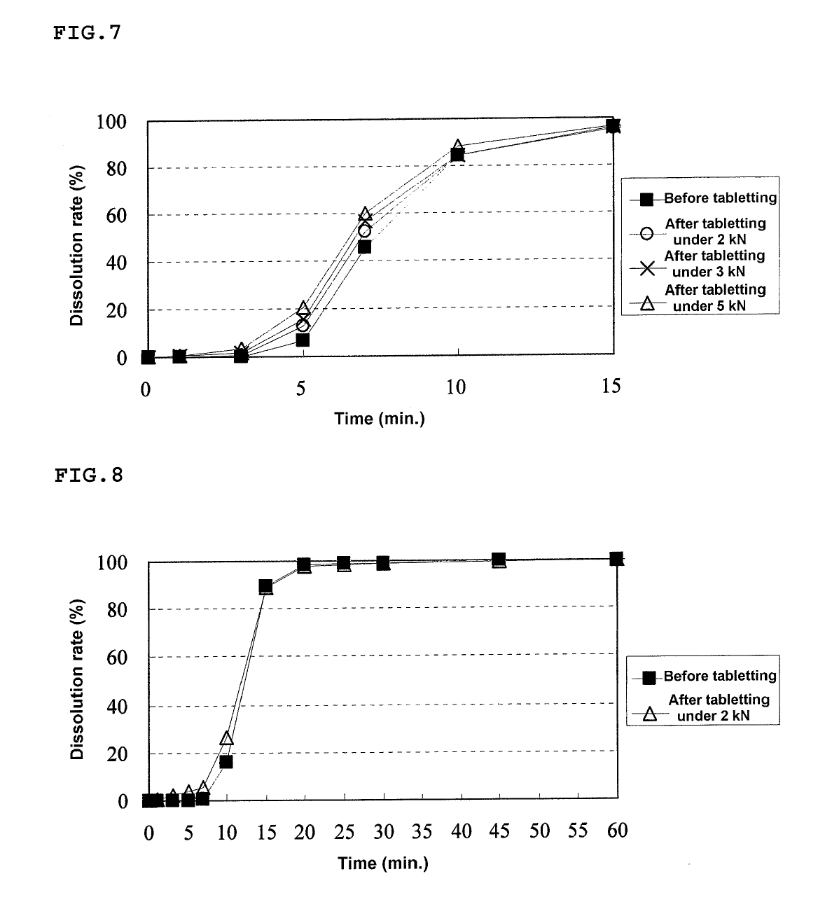

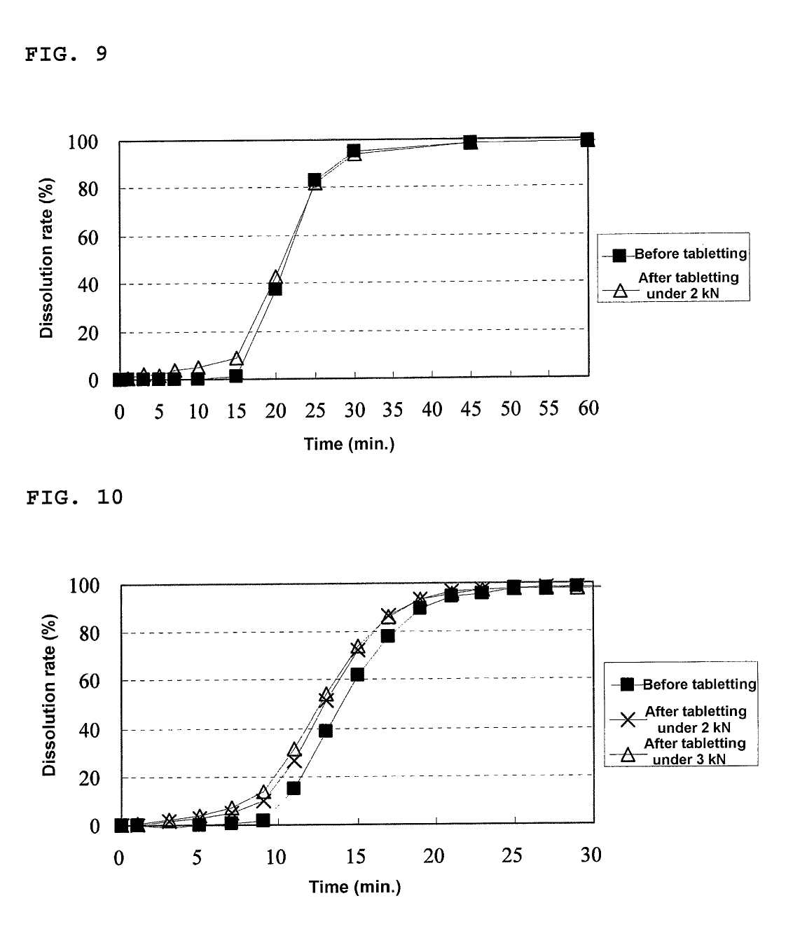

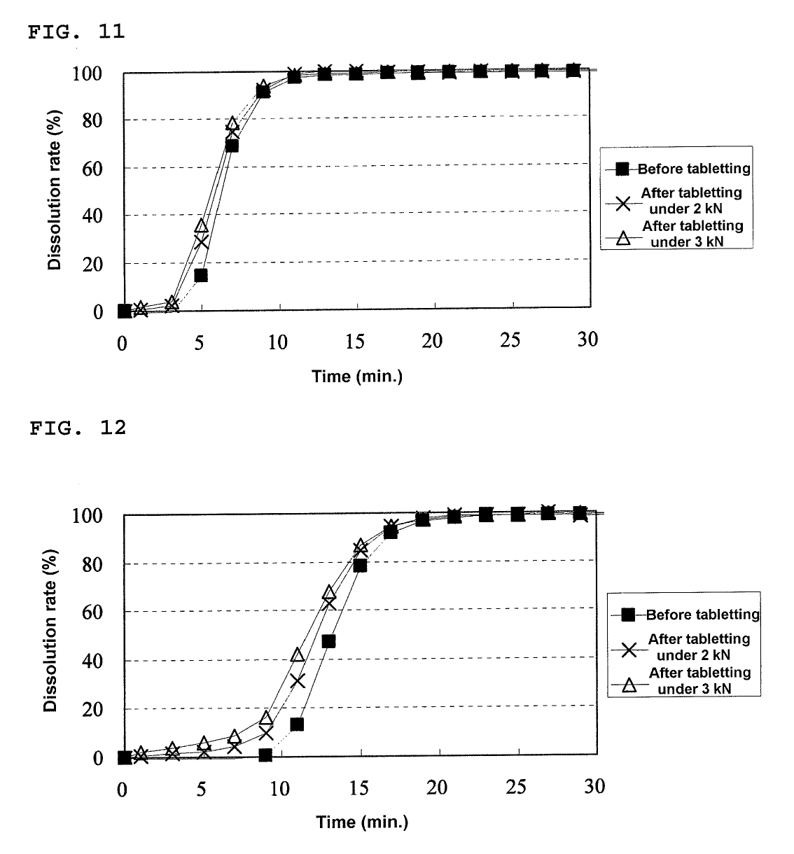

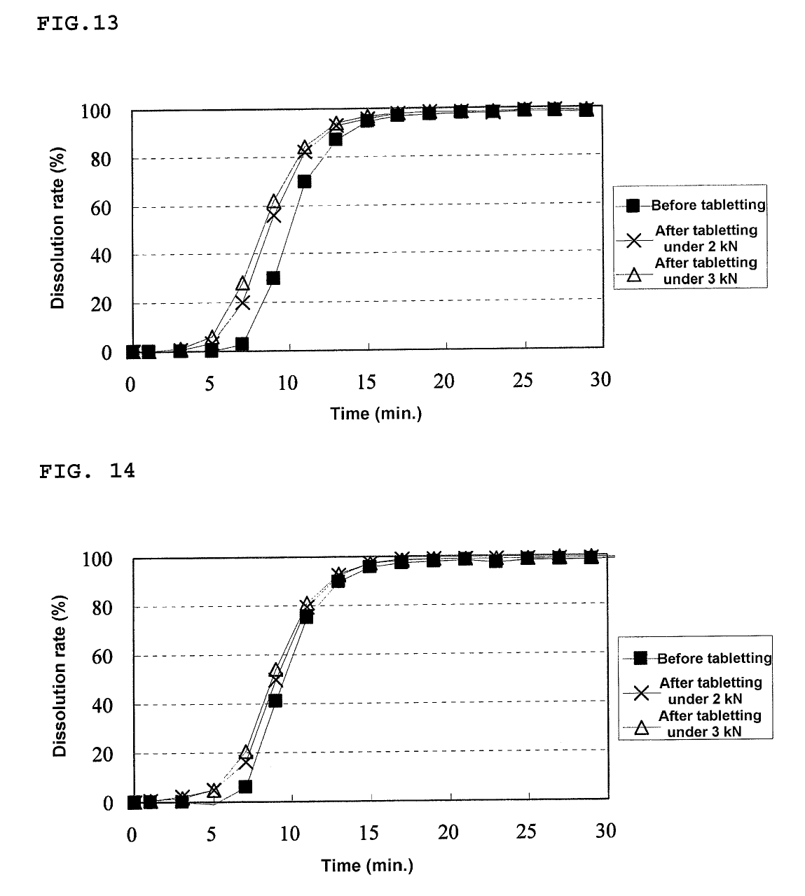

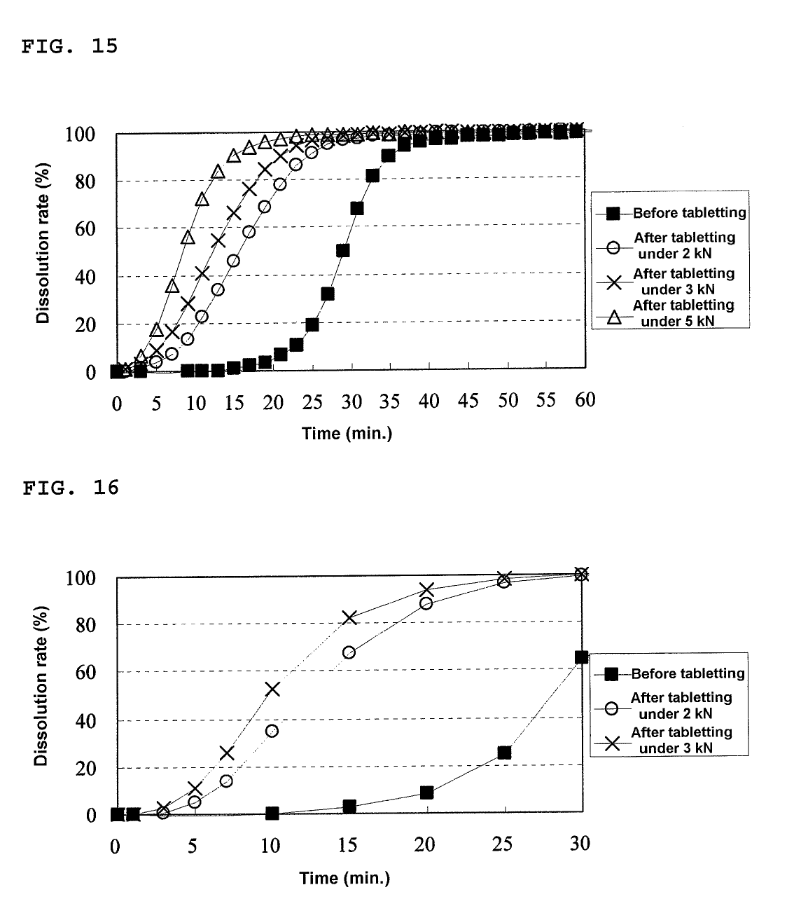

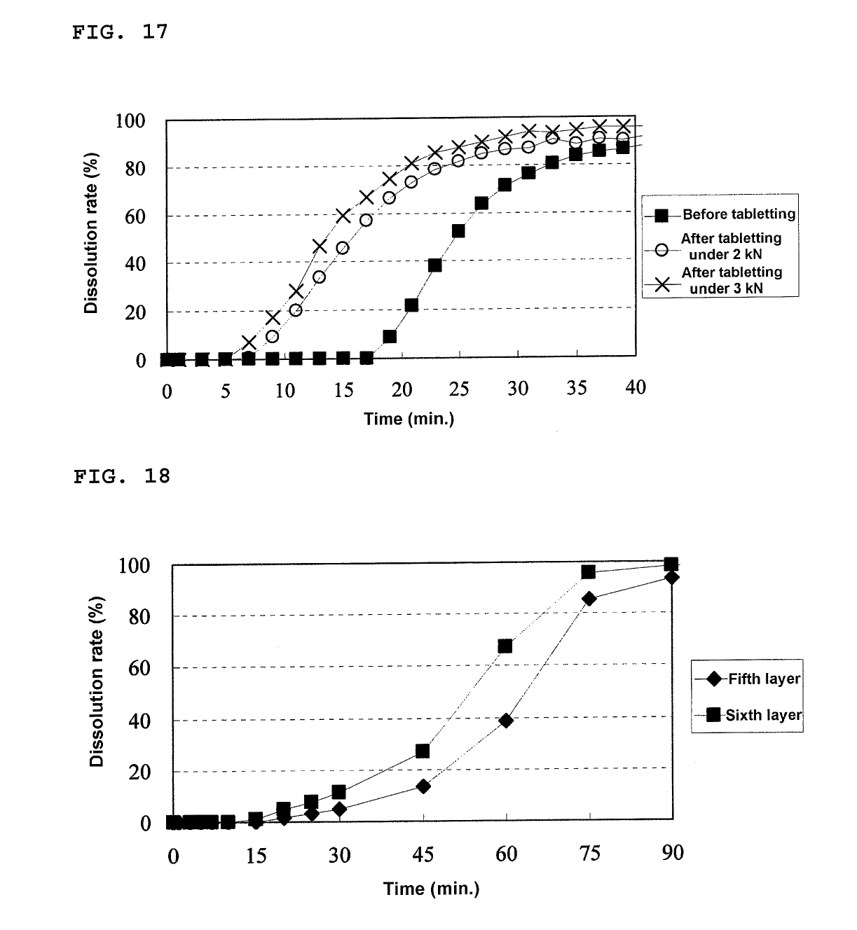

The resulting dissolution profiles are shown in FIGS. 1 to 18. T_{2%}, T_{50%}, D_{T2%}, D_{T50}%, D_{T2%} dissolution change, and D_{T50%} dissolution change are shown in Tables 28 to 36. T_{2%} represents the time when the dissolution rate reaches 2%. T_{50%} represents the time when the dissolution rate exceeds 50%. D_{T2%} represents the dissolution rate at the point of T_{2%} (before tabletting). D_{T50%} represents the dissolution rate at the point of T_{50%} (before tabletting). The changes in dissolution are calculated from the following equations:

D_{T2%} dissolution change (%)=D_{T2%}−2 (%)

D_{T50%} dissolution change (%)=D_{T5%}−50 (%)

TABLE 28

Example 4

| | 0 kN | 2 kN | 3 kN | 5 kN |
|---|---|---|---|---|
| Before tabletting, T_{2%} (min) | 1.0 | — | — | — |
| Before tabletting, T_{50%} (min) | 2.6 | — | — | — |
| After tabletting, D_{T2%} (min) | — | 3.6 | 5.4 | 5.5 |
| After tabletting, D_{T50%} (%) | — | 53.6 | 57.1 | 59.5 |
| D_{T2%} dissolution change (%) | — | 1.6 | 3.4 | 3.5 |
| D_{T50%} dissolution change (%) | — | 3.6 | 7.1 | 9.5 |

TABLE 29

| | Example 5 | | | | Example 6 | | | |
|---|---|---|---|---|---|---|---|---|
| | 0 kN | 2 kN | 3 kN | 5 kN | 0 kN | 2 kN | 3 kN | 5 kN |
| Before | 3.9 | — | — | — | 7.0 | — | — | — |
| tabletting, | | | | | | | | |
| T_{2%} (min) | | | | | | | | |
| Before | 7.1 | — | — | — | 11.1 | — | — | — |
| tabletting, | | | | | | | | |
| T_{50%} (min) | | | | | | | | |
| After | — | 5.7 | 6.7 | 9.1 | — | 4.5 | 7.4 | 10.9 |
| tabletting, | | | | | | | | |
| D_{T2%} (min) | | | | | | | | |
| After | — | 59.6 | 61.3 | 65.3 | — | 58.6 | 64.5 | 66.9 |
| tabletting, | | | | | | | | |
| D_{T50%} (%) | | | | | | | | |
| D_{T2%} | — | 3.7 | 4.7 | 7.1 | — | 2.5 | 5.4 | 8.9 |
| dissolution | | | | | | | | |
| change (%) | | | | | | | | |
| D_{T50%} | — | 9.6 | 11.3 | 15.3 | — | 8.6 | 14.5 | 16.9 |
| dissolution | | | | | | | | |
| change (%) | | | | | | | | |

TABLE 30

| | Example 7 | | | | Example 8 | | | |
|---|---|---|---|---|---|---|---|---|
| | 0 kN | 2 kN | 3 kN | 5 kN | 0 kN | 2 kN | 3 kN | 5 kN |
| Before | 0.4 | — | — | — | 1.2 | — | — | — |
| tabletting, | | | | | | | | |
| T_{2%} (min) | | | | | | | | |
| Before | 2.1 | — | — | — | 4.0 | — | — | — |
| tabletting, | | | | | | | | |
| T_{50%} (min) | | | | | | | | |
| After | — | 1.2 | 2.3 | 3.8 | — | 1.7 | 2.0 | 3.1 |
| tabletting, | | | | | | | | |
| D_{T2%} (min) | | | | | | | | |
| After | — | 49.6 | 51.4 | 54.3 | — | 46.3 | 50.4 | 51.8 |
| tabletting, | | | | | | | | |
| D_{T50%} (%) | | | | | | | | |
| D_{T2%} | — | −0.8 | 0.3 | 1.8 | — | −0.3 | 0.0 | 1.1 |
| dissolution | | | | | | | | |
| change | | | | | | | | |
| (%) | | | | | | | | |
| D_{T50%} | — | −0.4 | 1.4 | 4.3 | — | −3.7 | 0.4 | 1.8 |
| dissolution | | | | | | | | |
| change (%) | | | | | | | | |

TABLE 31

| | Example 9 | | | | Example 10 | | | |
|---|---|---|---|---|---|---|---|---|
| | 0 kN | 2 kN | 3 kN | 5 kN | 0 kN | 2 kN | 3 kN | 5 kN |
| Before | 3.1 | — | — | — | 3.6 | — | — | — |
| tabletting, | | | | | | | | |
| T_{2%} (min) | | | | | | | | |
| Before | 5.8 | — | — | — | 7.3 | — | — | — |
| tabletting, | | | | | | | | |
| T_{50%} (min) | | | | | | | | |
| After | — | 3.5 | 4.4 | 4.8 | — | 4.2 | 5.9 | 8.6 |
| tabletting, | | | | | | | | |
| D_{T2%} (min) | | | | | | | | |
| After | — | 55.8 | 56.7 | 55.7 | — | 55.5 | 59.4 | 63.0 |
| tabletting, | | | | | | | | |
| D_{T50%} (%) | | | | | | | | |
| D_{T2%} | — | 1.5 | 2.4 | 2.8 | — | 2.2 | 3.9 | 6.6 |
| dissolution | | | | | | | | |
| change | | | | | | | | |
| (%) | | | | | | | | |
| D_{T50%} | — | 5.8 | 6.7 | 5.7 | — | 5.5 | 9.4 | 13.0 |
| dissolution | | | | | | | | |
| change (%) | | | | | | | | |

TABLE 32

| | |
|---|---|
| Example 11 | Example 12 |

| | 0 kN | 2 kN | 0 kN | 2 kN |
|---|---|---|---|---|
| Before tabletting, T_{2%} (min) | 7.3 | — | 15.2 | — |
| Before tabletting, T_{50%} (min) | 12.3 | — | 21.4 | — |
| After tabletting, D_{T2%} (min) | — | 7.4 | — | 10.0 |
| After tabletting, D_{T50%} (%) | — | 55.4 | — | 53.9 |
| D_{T2%} dissolution change (%) | — | 5.4 | — | 8.0 |
| D_{T50%} dissolution change (%) | — | 5.4 | — | 3.9 |

TABLE 33

| | Example | | Example | | Example | |
|---|---|---|---|---|---|---|
| | 13 | | 14 | | 16 | |
| | 2 kN | 3 kN | 2 kN | 3 kN | 2 kN | 3 kN |
| D_{T2%} dissolution change (%) | 8.4 | 12.0 | 7.0 | 4.6 | 10.5 | 17.5 |
| D_{T50%} dissolution change (%) | 11.5 | 13.9 | 13.8 | 9.0 | 14.9 | 19.6 |

TABLE 34

| | |
|---|---|
| Example 17 | Example 18 |

| | 2 kN | 3 kN | 2 kN | 3 kN |
|---|---|---|---|---|
| D_{T2%} dissolution change (%) | 13.2 | 19.6 | 8.0 | 10.1 |
| D_{T50%} dissolution change (%) | 19.2 | 23.3 | 7.5 | 11.4 |

TABLE 35

| | Comparative Example 1 | | | | Comparative Example 2 | | | |
|---|---|---|---|---|---|---|---|---|
| | 0 kN | 2 kN | 3 kN | 5 kN | 0 kN | 2 kN | 3 kN | 5 kN |
| Before | 16.9 | | | | 13.9 | — | — | — |
| tabletting, | | | | | | | | |
| T_{2%} (min) | | | | | | | | |
| Before | 29.0 | | | | 28.2 | — | — | — |
| tabletting, | | | | | | | | |
| T_{50%} (min) | | | | | | | | |
| After | — | 57.1 | 75.3 | 93.8 | — | 60.6 | 76.0 | — |
| tabletting, | | | | | | | | |
| D_{T2%} (min) | | | | | | | | |
| After | — | 96.7 | 98.6 | 98.7 | — | 98.4 | 99.2 | — |
| tabletting, | | | | | | | | |
| D_{T50%} (%) | | | | | | | | |
| D_{T2%} | — | 55.1 | 73.3 | 91.8 | — | 58.6 | 74.0 | — |
| dissolution | | | | | | | | |
| change (%) | | | | | | | | |
| D_{T50%} | — | 46.7 | 48.6 | 48.7 | — | 48.4 | 49.2 | — |
| dissolution | | | | | | | | |
| change (%) | | | | | | | | |

TABLE 36

Comparative Example 3

| | 0 kN | 2 kN | 3 kN | 5 kN |
|---|---|---|---|---|
| Before tabletting, T_{2%} (min) | 17.5 | — | — | — |
| Before tabletting, T_{50%} (min) | 24.7 | — | — | — |
| After tabletting, D_{T2%} (min) | — | 59.2 | 68.8 | — |
| After tabletting, D_{T50%} (%) | — | 81.2 | 87.4 | — |
| D_{T2%} dissolution change (%) | — | 57.2 | 66.8 | — |
| D_{T50%} dissolution change (%) | — | 31.2 | 37.4 | — |

### Experimental Example 2

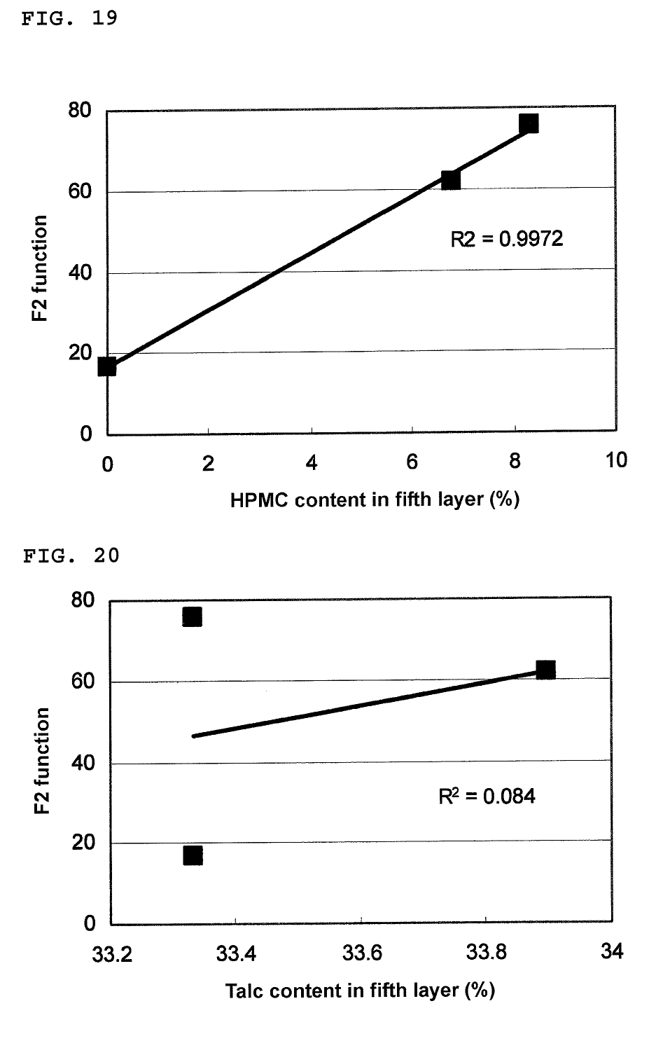

The f2 function is known as an index for evaluating the equivalence in dissolution behavior. The f2 function value is represented as the following equation:

$${f}_{2} = 50 log [ \frac{100}{\sqrt{1 + \frac{{\sum }_{i = 1}^{n} {( Ti - Ri )}^{2}}{n}}} ] \qquad [ Math . 1 ]$$

wherein Ti and Ri are average dissolution rates at each point in time of a test formulation and a standard formulation, respectively, and n is the number of the points to compare the average dissolution rates with each other. When the f2 value is 50 or more, it is judged that the test formulation is equivalent to the standard formulation [source: Iyakuhin Seizo Hanbai Shishin 2008 (DRUG APPROVAL AND LICENSING PROCEDURES IN JAPAN 2008), published by Jiho, Inc., 272-277]. With respect to the granular pharmaceutical compositions (before tabletting) prepared in Examples 5 and 8 and Comparative Example 1 and the rapidly disintegrating tablets in the buccal cavity containing the same (after tabletting under a pressure of 2 kN), a dissolution test was carried out using a 6-unit automatic dissolution test apparatus in accordance with the dissolution test, method 2 described in the Japanese Pharmacopoeia, and the obtained results were evaluated by the f2 function. These granular pharmaceutical compositions had the same formulation with respect to up to the fourth layer, but were different in the content of a water-soluble polymer contained in the fifth layer. The relationship between the content of a water-soluble polymer HPMC in the fifth layer and the f2 function is shown in FIG. 19, and the relationship between the content of a glidant, talc in the fifth layer and the f2 function is shown in FIG. 20. The f2 function was increased dependently on the HPMC content in the fifth layer, but the f2 did not change dependently on the talc content in the fifth layer. This result suggests that a decreased change in a release rate after compression-molding is achieved by the addition of the water-soluble polymer HPMC.

### INDUSTRIAL APPLICABILITY

The present invention relates to a granular pharmaceutical composition for oral administration, wherein a particle containing a drug having a bitter taste is coated with a coating comprising a methyl methacrylate-butyl methacrylate-dimethylaminoethyl methacrylate copolymer and a water-soluble polymer; a rapidly disintegrating tablet in the buccal cavity containing the granular pharmaceutical composition; and a use of a methyl methacrylate-butyl methacrylate-dimethylaminoethyl methacrylate copolymer and a water-soluble polymer in the manufacture of the granular pharmaceutical composition.

According to the present invention, unpleasantness caused by a drug having an unpleasant taste can be decreased to improve compliance. Drug release from the core of the granular pharmaceutical composition after compression-molding can be decreased. The drug is released in the upper gastrointestinal tract by being rapidly released after a certain period of time, to show a sufficient efficacy of the drug.

The present invention can be widely applied to drugs having various properties.

As above, the present invention was explained with reference to particular embodiments, but modifications and improvements obvious to those skilled in the art are included in the scope of the present invention.

1. A granular pharmaceutical composition for oral administration, wherein a drug-containing particle is coated with a coating comprising a methyl methacrylate-butyl methacrylate-dimethylaminoethyl methacrylate copolymer and a water-soluble polymer.

2. The granular pharmaceutical composition for oral administration according to claim 1, wherein the coating further comprises a glidant.

3. The granular pharmaceutical composition for oral administration according to claim 1, wherein the water-soluble polymer is one compound, or two or more compounds selected from the group consisting of hydroxypropyl cellulose, hydroxypropyl methylcellulose, methylcellulose, hydroxyethyl cellulose, povidone, copolyvidone, a polyvinyl alcohol-polyethylene glycol graft copolymer, polyvinyl alcohol, macrogol, and polyethylene oxide.

4. The granular pharmaceutical composition for oral administration according to claim 1, wherein the water-soluble polymer is one compound, or two or more compounds selected from the group consisting of hydroxypropyl cellulose, hydroxypropyl methylcellulose, methylcellulose, and hydroxyethyl cellulose.

5. The granular pharmaceutical composition for oral administration according to claim 1, wherein an amount of the water-soluble polymer is 1% by weight to 30% by weight with respect to an amount of the methyl methacrylate-butyl methacrylate-dimethylaminoethyl methacrylate copolymer.

6. The granular pharmaceutical composition for oral administration according to claim 2, wherein the glidant is one compound, or two or more compounds selected from the group consisting of metal silicates, silicon dioxides, higher fatty acid metal salts, metal oxides, alkaline earth metal salts, and metal hydroxides.

7. The granular phamaceutical composition for oral administration according to claim 2, wherein the glidant is one compound, or two or more compounds selected from the group consisting of talc, kaolin, calcium silicate, magnesium silicate, light anhydrous silicic acid, magnesium stearate, calcium stearate, iron oxide, titanium oxide, calcium carbonate, calcium phosphate, gypsum, magnesium carbonate, aluminum hydroxide, hydrated silicon dioxide, microcrystalline cellulose, synthetic aluminum silicate, heavy anhydrous silicic acid, aluminum magnesium hydroxide, stearic acid, corn starch, magnesium aluminate metasilicate, dibasic calcium phosphate fine granulated, and glyceryl monostearate.

8. The granular pharmaceutical composition for oral administration according to claim 2, wherein the glidant is one compound, or two or more compounds selected from the group consisting of talc, kaolin, calcium silicate, magnesium silicate, light anhydrous silicic acid, magnesium stearate, and glyceryl monostearate.

9. The granular pharmaceutical composition for oral administration according to claim 2, wherein an amount of the glidant is 1% by weight to 500% by weight with respect to an amount of the methyl methacrylate-butyl methacrylate-dimethylaminoethyl methacrylate copolymer.

10. The granular pharmaceutical composition for oral administration according to claim 1, wherein the water-soluble polymer is hydroxypropyl methylcellulose, and an amount of hydroxypropyl methylcellulose is 1% by weight to 30% by weight with respect to an amount of the methyl methacrylate-butyl methacrylate-dimethylaminoethyl methacrylate copolymer.

11. The granular pharmaceutical composition for oral administration according to claim 1, wherein the drug is an acidic drug or a salt thereof.

12. The granular pharmaceutical composition for oral administration according to claim 1, wherein the drug has an unpleasant taste.

13. The granular pharmaceutical composition for oral administration according to claim 1, wherein (1) a coating layer containing (i) a coating consisting of a methyl methacrylate-butyl methacrylate-dimethylaminoethyl methacrylate copolymer and a water-soluble polymer, or (ii) a coating consisting of a methyl methacrylate-butyl methacrylate-dimethylaminoethyl methacrylate copolymer, a water-soluble polymer, and a glidant surrounds (2) a layer comprising one, or two or more water-soluble insolubilizers and one, or two or more water-soluble insolubilizing substances.

14. A rapidly disintegrating tablet in the buccal cavity, comprising the granular pharmaceutical composition for oral administration according to claim 1.

15. A process of manufacturing a granular pharmaceutical composition for oral administration, characterized by coating a drug-containing particle with a coating comprising a methyl methacrylate-butyl methacrylate-dimethylaminoethyl methacrylate copolymer and a water-soluble polymer.

16. The process according to claim 15, wherein the coating further comprises a glidant.

17. The process according to claim 15, wherein the water-soluble polymer is one compound, or two or more compounds selected from the group consisting of hydroxypropyl cellulose, hydroxypropyl methylcellulose, methylcellulose, hydroxyethyl cellulose, povidone, copolyvidone, a polyvinyl alcohol-polyethylene glycol graft copolymer, polyvinyl alcohol, macrogol, and polyethylene oxide.

18. The process according to claim 16, wherein the glidant is one compound, or two or more compounds selected from the group consisting of metal silicates, silicon dioxides, higher fatty acid metal salts, metal oxides, alkaline earth metal salts, and metal hydroxides.

19. A process of manufacturing the granular pharmaceutical composition for oral administration according to claim 1, comprising the steps of:
(1) forming a layer comprising a water-soluble insolubilizer and a water-soluble insolubilizing substance outside a drug-containing particle, and
(2) coating the obtained particle with (i) a coating consisting of a methyl methacrylate-butyl methacrylate-dimethylaminoethyl methacrylate copolymer and a water-soluble polymer, or (ii) a coating consisting of a methyl methacrylate-butyl methacrylate-dimethylaminoethyl methacrylate copolymer, a water-soluble polymer, and a glidant.

20. (canceled)
